(19) United States
(12) Patent Application Publication
(10) Pub. No.: US 2025/0167998 A1
(43) Pub. Date: May 22, 2025

(54) PERSONAL MEDICAL AVATAR

(71) Applicant: CURELATOR, INC., Palmer, MA (US)

(72) Inventors: Alec MIAN, Palmer, MA (US); Stephen DONOGHUE, Palmer, MA (US); Marc ALBERT, Palmer, MA (US)

(21) Appl. No.: 18/839,146

(22) PCT Filed: Feb. 20, 2023
(86) PCT No.: PCT/US2023/062908

§ 371 Date: Aug. 16, 2024

Related U.S. Application Data
(60) Provisional application No. 63/268,225, filed on Feb. 18, 2022.

(51) Int. Cl. H04L 9/14; G06T 13/40; G06T 13/80; G16H 10/60; G16H 40/67; G16H 50/50; G16H 80/00

(52) U.S. Cl. CPC H04L 9/14; G06T 13/40; G06T 13/80; G16H 10/60; G16H 40/67; G16H 50/50; G16H 80/00; G06T 2200/24; G06T 2210/41

(57) ABSTRACT

A method of interacting with a patient avatar is provided that includes receiving patient data, including historical health information, for at least one patient at a centralized server system associated with at least one of a clinician device or a. patient device. The method also includes generating the patient avatar based on the patient data. The patient avatar includes a two-dimensional or three-dimensional graphical representation of the patient, with the historical health information displayed as physical characteristics of the patient avatar. The method additionally includes displaying the patient avatar on a graphical user interface associated with at least one of the clinician device or the patient device. The method further includes playing back a graphical representation of a temporal evolution of the patient avatar in response to receiving a playback command. The temporal evolution of the patient avatar includes a change overtime in historical health information of the patient.

### CROSS-REFERENCE TO RELATED APPLICATIONS

This application incorporates by reference in its entirety the disclosure of U.S. provisional application 63/268,225 filed on Feb. 18, 2022; and U.S. provisional application 63/363,774, filed Apr. 28, 2022.

### BACKGROUND

Wearable devices and other data entry devices have made it increasingly possible for patients to digitize, transfer, store, analyze and share their data and activities on-line. In some instances, data can be used to create avatars. Previously, avatars could be created to resemble the physical characteristics of a patient. However, those avatars depended on data entered by the patient and only the most recent data entered was stored. Thus, such avatars could only be as accurate as the data the patient had entered. Additionally, current avatars are typically static. For example, the physical characteristics of the avatars typically stay the same and the avatar does not move or send alerts to the patient, and in particular is incapable of displaying accurate data gathered over a temporal period.

There is a need in this art for improvements to digital, real-time, patient data repository entities that are clinically accurate, anthropomorphized animations of the patient's disease state as well as the patient's longitudinal patient experiences.

### SUMMARY

This disclosure relates generally to generating a patient avatar based on historical health information and to at least one of a patient or a clinician interacting with the patient avatar.

A first embodiment describes a method of interacting with a patient avatar that includes receiving patient data for at least one patient at a centralized server system associated with at least one of a clinician device or a patient device, where the patient data includes historical health information. The method includes generating, based on the patient data for the at least one patient, the patient avatar. The patient avatar includes a two-dimensional or three-dimensional graphical representation of the at least one patient, and the historical health information is visually displayed as physical characteristics of the patient avatar. The method also includes displaying the patient avatar on a graphical user interface associated with at least one of the clinician device or the patient device. Displaying the patient avatar includes displaying most recent patient data based on the historical health information. The method further includes playing back a graphical representation of a temporal evolution of the patient avatar in response to receiving a playback command from at least one of the clinician device or the patient device. The temporal evolution of the patient avatar includes a change over time in historical health information of the at least one patient.

Within examples, a second embodiment describes a method of interacting with a patient avatar by a clinician that includes receiving patient data for at least one patient at a server system associated with a clinician device, where the patient data includes historical health information. The method includes generating the patient avatar based on the patient data for the at least one patient. The patient avatar includes a two-dimensional or three-dimensional graphical representation of the at least one patient, and the historical health information is visually displayed as physical characteristics of the patient avatar. The method also includes displaying the patient avatar on a graphical user interface associated with the clinician device. Displaying the patient avatar includes displaying most recent patient data based on the historical health information. The method further includes playing back a graphical representation of a temporal evolution of the patient avatar in response to receiving a playback command from the clinician device, where the temporal evolution of the patient avatar includes a change over time in historical health information of the at least one patient.

Within examples, a third embodiment describes a method of generating a patient avatar that includes receiving patient data for at least one patient at a server system associated with a patient device, where the patient data includes historical health information. The method includes generating the patient avatar based on the patient data for the at least one patient. The patient avatar includes a two-dimensional or three-dimensional graphical representation of the at least one patient, and the historical health information is visually displayed as physical characteristics of the patient avatar. The method also includes displaying the patient avatar on a graphical user interface associated with the patient device, where displaying the patient avatar includes displaying most recent patient data based on the historical health information. The method further includes playing back a graphical representation of a temporal evolution of the patient avatar in response to receiving a playback command from the patient device, where the temporal evolution of the patient avatar includes a change over time in historical health information of the at least one patient.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE DRAWINGS

FIG. 1 illustrates a centralized server in communication with a patient device and a clinician device, according to an example embodiment.

FIG. 2 illustrates an example method, according to an example embodiment.

FIG. 3A illustrates a patient avatar and GUI, according to an example embodiment.

FIG. 3B illustrates a patient avatar alert, according to an example embodiment.

FIG. 3C illustrates a patient avatar alert, according to an example embodiment.

FIG. 3D illustrates a patient avatar alert, according to an example embodiment.

FIG. 3E illustrates a patient avatar alert, according to an example embodiment.

FIG. 3F illustrates a patient avatar alert, according to an example embodiment.

FIG. 3G illustrates a patient avatar alert, according to an example embodiment.

FIG. 3H illustrates a patient avatar alert, according to an example embodiment.

FIG. 3I illustrates a patient avatar alert, according to an example embodiment.

FIG. 4A illustrates a patient avatar comparison, according to an example embodiment.

FIG. 4B illustrates a patient avatar and an additional avatar comparison, according to an example embodiment.

FIG. 5 illustrates a temporal evolution of a patient avatar, according to an example embodiment.

FIG. 6A illustrates a question prompt, according to an example embodiment.

FIG. 6B illustrates a question prompt, according to an example embodiment.

FIG. 7A illustrates a real-time conversation request, according to an example embodiment.

FIG. 7B illustrates a real-time conversation request, according to an example embodiment.

FIG. 8 illustrates an example method, according to an example embodiment.

FIG. 9 illustrates an example method, according to an example embodiment.

FIG. 10A illustrates a prompt for the user to answer a question, according to an example embodiment.

FIG. 10B illustrates an informational prompt, according to an example embodiment.

FIG. 11A illustrates a flowchart of entering symptom data, according to an example embodiment.

FIG. 11B illustrates a graphical user interface for inputting symptom data, according to an example embodiment.

FIG. 11C illustrates a graphical user interface for inputting symptom data, according to an example embodiment.

### DETAILED DESCRIPTION

Example methods and systems of the disclosed invention are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, can be arranged, substituted, combined, separated, and designed in a wide variety of different configurations, all of which are explicitly contemplated herein.

Currently, health data can be collected from a patient and stored in a digital format. For example, health data can be collected through wearable devices or through user questionnaires via a graphical user interface. That health data can also be used to generate an avatar of a patient. Typically, the avatars generated from the health data display the patient according to the most current health data. For example, when a patient's weight is received, and the avatar can update its apparent weight to resemble the current weight. Avatars can also be used to enter symptomatic inflictions. For example, the patient can enter that they have a runny nose, and the avatar could display the runny nose. These avatars typically update with addition of new data, to show the patient's current health state. Some embodiments can even use data gathered to predict a future health state of the patient. However, avatars previously described do not provide a way to efficiently view data acquired over a temporal period and visualize the evolution thereof in a way that is easy to digest. In contrast, provided herein are embodiments of the disclosed invention that can archive historical health information and animate patient data on an avatar at different play back speeds to illustrate the progression of the patient's health over a temporal period. This feature allows a healthcare professional to accurately diagnose the patient based on the progression of the patient's medical history and symptom evolution.

In a present embodiment, interaction with a patient avatar that animates progression of a patient's disease symptoms over a temporal period can be achieved. Patient data including historical health information for a patient can be received at a centralized server. The centralized server can be in communication with one or both of a patient device and a clinician device. Based on the patient data, a patient avatar can be generated at the centralized server. The patient avatar can be a two-dimensional or three-dimensional graphical representation of the patient with physical characteristics of the patient avatar representing the historical health information. Once the patient avatar is generated at the centralized server, it can be sent to the patient device and/or the clinician device. In an alternative embodiment, the patient avatar can be generated at the patient device and then sent via the centralized server to the clinician device. Additionally, the avatar could be generated at the clinician device and then sent via the centralized server to the patient device.

Once generated, the patient avatar can be displayed in a graphical user interface (GUI) of the clinician device and/or the patient device. A user can view the patient avatar on the patient device simultaneously or separately from a second user viewing the patient avatar on the clinician device. The displayed patient avatar can display a visual representation of the patient the most recent patient data with the physical characteristics of the patient avatar. For example, if the patient most recently experienced neck pain and sensitivity to light, the patient avatar could be displayed with shading on a neck area of the patient avatar and stars on or near patient avatar eyes.

When the patient avatar is displayed on the GUI of the patient device and/or the clinician device, a user can select a play back command from the GUI. In response to receiving the playback command, the method can include playing back a graphical representation of a temporal evolution of the patient avatar. In one embodiment the temporal evolution of the patient avatar is a change overtime in historical health information of the patient. For example, the historical health information can be gathered from the patient over two months. The patient avatar can play out the evolution of the patient's patient data over two months. In an example embodiment, the patient avatar could indicate menstrual cycle over the two months, days with headaches over the two months, light sensitivity over the two months, and any other data, all on the physical representation of the patient—the patient avatar. Via the GUI of the patient device and/or the clinician device, the user can change the speed of the evolution and can stop on specific days. From the specific days, additional patient data can be accessed. These features of the disclosed invention permit a healthcare professional to accurately diagnose the patient based on the progression of the patient's medical history and symptom evolution in the absence of the presence of the patient herself to the healthcare professional.

As described herein, some embodiments of interacting with a patient avatar may include receiving patient data for at least one patient at a server system associated with a clinician device, where the patient data includes historical health information. The patient avatar can then be generated based on the patient data for the at least one patient. As previously stated, the patient avatar could be generated by at least one of the centralized server, the patient device, and the clinician device. The patient avatar can include a two-dimensional or three-dimensional graphical representation of the at least one patient, and the historical health information can be visually displayed as physical characteristics of the patient avatar. Once generated, the patient avatar can be sent to the clinician device and/or the patient device and displayed on a GUI associated with the respective clinician device and/or patient device. Displaying the avatar can include displaying most recent patient data based on the historical health information. In response to receiving a playback command the clinician device and/or the patient device, the GUI can play back a graphical representation of a temporal evolution of the patient avatar, where the temporal evolution of the patient avatar includes a change over time in historical health information of the at least one patient.

As used herein, historical health information is data related to a patient. For example, historical health information includes health records gathered from a patient's medical professional. Historical health information also includes answers to health questionnaires filled out by the patient, and information gathered from wearable devices. Historical health information can be compiled for years and archived in the patient avatar.

As used herein, a disease symptom is an observable manifestation of a particular disease or disorder. In operation, a disease symptom can be characterized by multiple characterization metrics, including but not limited to one or more of: (i) a time (or range of times) when the user experienced the disease symptom; (ii) a severity of the disease symptom; (iii) aspects or characteristics describing the disease symptom; and/or (iv) whether the disease symptom was accompanied by other related disease symptoms (and perhaps disease factors and/or disease triggers, which are described in further detail below). In non-limiting examples, where the disease is migraine and the disease symptom is a migraine headache, the characterization metrics for the migraine headache symptom can include any one or more of: (i) when the headache occurred; (ii) how long the headache lasted; (iii) the intensity and/or severity of the headache; (iv) the location of the headache along the user's head; and/or (v) whether the headache was accompanied by other related symptoms such as nausea or dizziness, and if so, the time, duration, intensity/severity of the accompanying symptoms. Disease symptoms for other chronic diseases can include different characterization metrics. More generally, a symptom can be a physical or physiological manifestation that is not necessarily associated with a particular disease or disorder. For example, a user of a healthcare management application can experience a symptom without knowing, which, if any, known disease or disorder is causing the symptom.

As used herein, a disease factor is any event, exposure, action, or conduct related to and/or performed or experienced by a user that has the potential to influence, affect, or cause the user to experience a disease symptom, or in some cases, prevent the user from experiencing a disease symptom. Disease factors can include both: (i) voluntary or modifiable conduct and/or experiences by the user over which the user has at least some control, such as emotional states (anger, boredom, stress, anxiety, etc.), consumption of a particular food product, ingestion of a particular therapeutic agent, application of a particular therapeutic agent, ingestion of a particular dietary supplement or drug, performance of a particular physical activity, and/or exposure to a particular chemical agent; and (ii) involuntary or un-modifiable conduct and/or experiences, such as exposure to environmental factors (e.g., smog, sunlight, rain, snow, high or low humidity, or high or low temperatures), ingestion or other exposure to mandatory therapeutic agents or drugs (e.g., drugs to treat or maintain other diseases), and effects of other diseases or physical conditions over which the user has little or perhaps effectively no control.

Like a disease symptom, a disease factor can also be characterized by multiple characterization metrics, and different disease factors can have different characterization metrics. For example, for a food or drug consumption-based disease factor, the characterization metrics can include, for example: (i) when the user consumed the food or drug; and/or (ii) how much of the food or drug the user consumed. Characterization metrics for an exposure-based disease factor can include, for example: (i) when the user was exposed: (ii) the intensity (e.g., bright sunlight) of the exposure; and/or (iii) the duration of the exposure.

In some embodiments, disease factors can also include premonitory symptoms or warning signs that do not actually cause the user to experience a disease symptom but are closely associated with onset of a disease symptom for a particular user. Thus, disease symptoms may be associated with one or more premonitory symptoms. For the migraine example, a premonitory symptom might be a craving for sweet foods before the user experiences the migraine headache. In this example, the sweet craving does not cause the migraine, but instead is likely related to some physiological change associated with the migraine attack.

In some instances, a particular physical manifestation felt by the user can be a disease symptom or a disease factor depending on its position in the physiological pathway. To use obesity as an example, reduced physical activity can be a disease factor because it tends to cause a disease symptom associated with obesity (e.g., excess fat stores). But in a different pathway, reduced physical activity can be a disease symptom that is caused by, for example, osteoarthritis that often follows obesity due to increased fat mass.

In some contexts, symptoms can be observed in a user that are not associated with a particular disease. For example, a user can wish to track a particular symptom that she has identified as her “most bothersome symptom” (i.e., a symptom that the user feels is most disruptive or distracting out of a set of symptoms that the user might experience). In these examples, a user can track the symptom itself, along with potential factors that can be associated with the symptom. In this manner, statistical associations can be formed between various symptoms and factors without each symptom necessarily corresponding to a particular disease for an individual. Accordingly, as used herein, the term “symptom factor” can be any factor which relates to a symptom of a specific disease, but which precedes the ‘main’ or ‘most bothersome’ symptom. For example, “symptom factor” can be related to or include any event, exposure, action, or conduct related to and/or performed or experienced by a user that can influence, affect, or cause the user to experience a symptom, or in some cases, prevent the user from experiencing a symptom, without the symptom necessarily being associated with a particular disease or disorder. It should be understood that the disease factors described herein and defined above can more generally be referred to as symptom factors. Accordingly, any forthcoming description that involves disease factors as they pertain to disease symptoms can more generally be understood as symptom factors and corresponding symptoms. In other words, symptom factors may be the factors that contribute to causing a corresponding symptom of a disease.

A disease trigger is a disease factor that has been determined, for example through statistical analyses or other methods, to have a sufficiently strong association with a particular disease symptom for an individual user so as to become user-specific information of high interest and clinical use to the user and their clinician. In some cases, a disease trigger can be strongly associated with causing the user to experience the particular disease symptom, or at least increasing the risk or likelihood that the user will experience the particular disease symptom. In other cases, its opposite, a disease protector, can be strongly associated with preventing the user from experiencing the particular disease symptom, or at least decreasing the risk or likelihood that the user will experience the particular disease symptom; such disease triggers can be referred to herein as protectors because they tend to reduce the user's likelihood of experiencing the disease symptom. In some embodiments, a disease trigger for a user is a disease factor having a determined univariate association with a disease symptom for the user, where the determined univariate association demonstrates a statistically significant hazard ratio or odds ratio (greater than 1) or equivalent regression coefficient (greater than 0) at a fixed significance level (e.g., p-value less than 0.05) in univariate regression models or in equivalent multivariable models.

In some embodiments, one or more server systems analyze disease symptom and disease factor data received from a patient population to determine which disease factors rise to the level of disease triggers for a particular patient. In operation, a patient population may include many (hundreds, thousands, or perhaps millions) of patients who all share one or more similarities (e.g., the same age or age range, same gender, same ethnicity, same national origin, suffer from the same disease, have the same allergies, have the same genetic markers, and/or perhaps other similarities). Some patients may be members of multiple patient populations.

Some embodiments generally apply a two-step iterative approach to identify disease factors and triggers for a patient population, and then (based on the identified disease factors and triggers for the patient population) identify disease factors and triggers for an individual patient. For the first step, the server systems collect and analyze disease factor and disease trigger data from patients in a patient population to identify the disease factors that tend to be most strongly associated with a particular disease symptom for the patients in the patient population. Client devices (under direct or indirect control of the server systems) are configured to prompt patients in the patient population to enter characterization metrics for the disease factors that the server systems have determined to be most strongly associated with the particular disease symptom for the patient population. For the second step, the server systems analyze the disease factor characterization metrics for the patients in the patient population, and for each patient in the population, the server systems determine the strength of the association (for that patient) between particular disease factors and the disease symptom. Then, for each patient, the server systems designate the disease factors that are most strongly associated with the disease symptom as disease triggers for the patient. The process is iterative in that disease triggers identified for one patient in a patient population can be analyzed for the whole patient population and then tested for individual patients. Some aspects of this iterative, two-phase process are described in PCT Application PCT/US2014/013894 filed on Jan. 30, 2014, the contents of which are incorporated herein by reference.

For most users, many commonly suspected disease factors have turned out to not be disease triggers, i.e., the most suspected disease factors are neither triggers nor protectors for the user. However, there is a high degree of heterogeneity between users with respect to which disease factors affect different users as either disease triggers or disease protectors. To help a user manage his or her disease, the user advantageously wants individualized information about which suspected disease factors are disease triggers/protectors for them and which suspected disease factors have no effect for them. A suspected disease factor that has no effect for that individual is referred to herein as a “no-association factor” or NAF.

As used herein, a phase of a particular symptom can refer to a discrete period in which a particular physical manifestation or manifestations are expected in relation to a given symptom. Some symptoms may manifest in a cyclical manner denoted by a plurality of recurring phases. For example, migraine headaches, Irritable Bowel Syndrome (IBS), and asthma each have episodic recurrences characterized by two or more phases of experiencing the symptom. In the context of the present disclosure, experiencing a disease symptom can be understood as experiencing a level of severity of the disease symptom, and can include a level of severity relative to prior periods of experiencing the symptom. For example, this phenomenon can manifest as experiencing a worsening or improvement in the disease symptom relative to another phase or a past cycle. Other symptoms denoted by such episodic recurring phases are contemplated in the context of the following disclosure. As an example, related to recurring phases, an interictal phase between migraine attacks can typically be associated with no manifestations of a migraine headache or corresponding disease symptoms. However, as described below, such phases might not be tied to biomarker levels in any discrete way. For many symptoms, different phases can be sequential and cyclical, and can be predictable in terms of order (e.g., phase 1 always precedes phase 2, etc.) even if they are less predictable in terms of duration (e.g., how long phase 3 will last for a particular cycle).

In some embodiments described herein, a biomarker can be used to predict the occurrence of symptoms. A biomarker can refer to a measurable substance in a user, or physiological state of the user, or a perceived stressor experienced by the user, or a specific combination of these, whose presence (or a degree thereof) is indicative of one or more phenomena experienced by the user. For example, a “substance” in this context can be a detectable molecule produced by the human body that can be measured using a sensor device. A “physiological state” can include a condition or state of the body or bodily functions of a user. A “stressor” can be an emotional or physical prompt experienced by a user or a physiological response to such a prompt. For example, a “stressor” in the context of a migraine can include one or more of stress, anxiety, heart rate variability, breathing pattern, hearing sensitivity, and sleep quality. Other examples of biomarkers are possible as well. A biomarker can be more broadly understood as including biological signs of a given disease or condition. Within examples described herein, a biomarker level, and observed patterns in such biomarker levels during phases of a symptom, can be leveraged to predict the symptom and to recommend an intervention to prevent or mitigate the predicted symptom. Different biomarkers or combinations of biomarkers can be predictive of a symptom for different users.

The aforementioned factors, symptoms, and other definitions can all be included in the patient data that is received at the centralized server for generating the patient avatar.

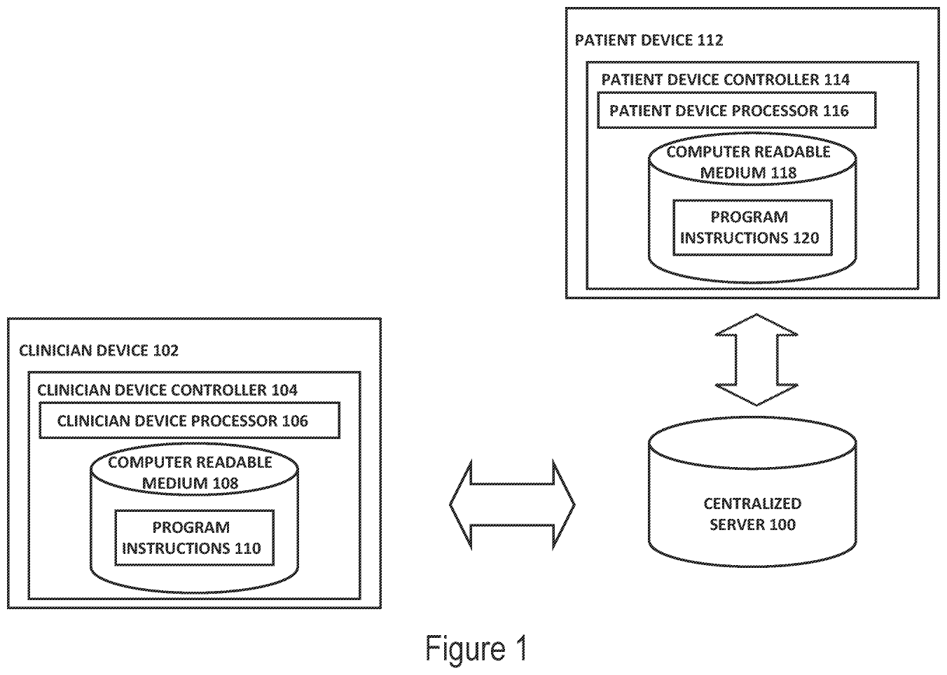

FIG. 1 is a simplified block diagram exemplifying a centralized server 100 associated with a clinician device 102 and/or a patient device 112 for receiving patient data for at least one patient, wherein the patient data comprises historical health information.

As illustrated in FIG. 1, the clinician device 102 may include a clinician device controller 104. Clinician device controller 104 includes at least one clinician device processor(s) 106, at least one clinician device analog to digital converter, and a clinician device memory. The clinician device memory may include a computer readable medium, such as a clinician device non-transitory computer readable medium 108, which may include without limitation, read-only memory (ROM), programmable read-only memory (PROM), erasable programmable read-only memory (EPROM), electrically erasable programmable read-only memory (EEPROM), non-volatile random-access memory (e.g., flash memory), a solid state drive (SSD), a hard disk drive (HDD), a Compact Disc (CD), a Digital Video Disk (DVD), a digital tape, read/write (R/W) CDs, R/W DVDs, etc. Other types of storage devices, memories, and media are also contemplated herein.

The clinician device non-transitory computer-readable medium 108 may also store a set of clinician device program instructions 110 executable by the clinician device processor(s) 106 to perform a plurality of clinician device operations. The at least one clinician device processor(s) 106 can include one or more processors, such as one or more general-purpose microprocessors and/or one or more special purpose microprocessors. The one or more processors may include, for instance, an application-specific integrated circuit (ASIC) or a field-programmable gate array (FPGA). Other types of processors, computers, or devices configured to carry out software instructions are also contemplated herein.

As further illustrated in FIG. 1, the patient device 112 may include a patient device controller 114. Patient device controller 114 includes at least one patient device processor(s) 116, at least one patient device analog to digital converter, and a patient device memory. The patient device memory may include a computer readable medium, such as a patient device non-transitory computer readable medium 118, which may include without limitation, read-only memory (ROM), programmable read-only memory (PROM), erasable programmable read-only memory (EPROM), electrically erasable programmable read-only memory (EEPROM), non-volatile random-access memory (e.g., flash memory), a solid state drive (SSD), a hard disk drive (HDD), a Compact Disc (CD), a Digital Video Disk (DVD), a digital tape, read/write (R/W) CDs, R/W DVDs, etc. Other types of storage devices, memories, and media are also contemplated herein.

The patient device non-transitory computer-readable medium 118 may also store a set of patient device program instructions 120 executable by the patient device processor(s) 116 to perform a plurality of patient device operations. The at least one patient device processor(s) 116 can include one or more processors, such as one or more general-purpose microprocessors and/or one or more special purpose microprocessors. The one or more processors may include, for instance, an application-specific integrated circuit (ASIC) or a field-programmable gate array (FPGA). Other types of processors, computers, or devices configured to carry out software instructions are also contemplated herein.

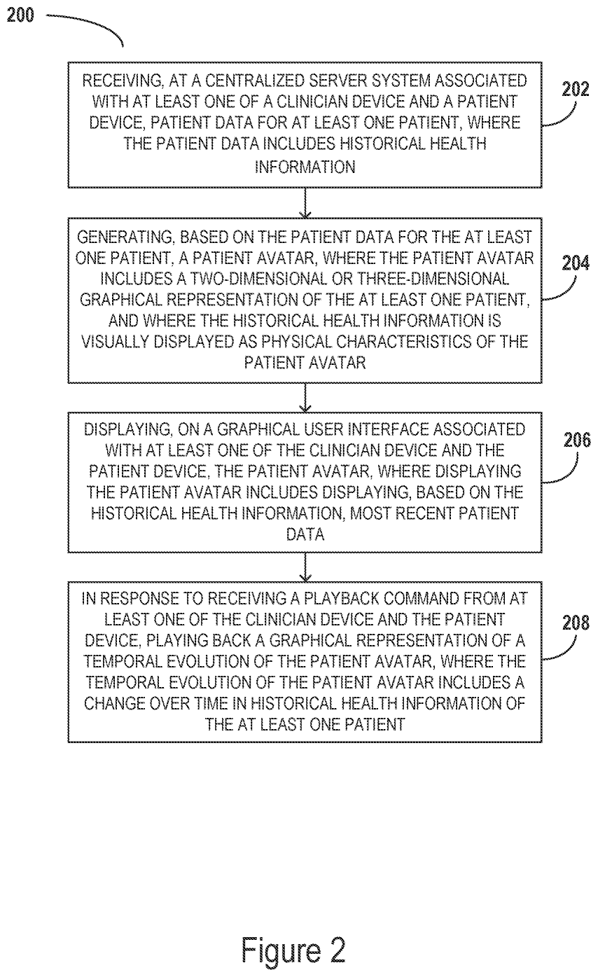

FIG. 2 is a flowchart of a method of interacting with a patient avatar, according to one or more example embodiments. Method 200 may include one or more operations, functions, or actions, as depicted by one or more of blocks 202, 204, 206, and 208 each of which may be carried out by any of the systems shown in prior figures, among other possible systems.

Those skilled in the art will understand that the flow charts described herein illustrate functionality and operation of certain implementations of the present disclosure. In this regard, each block of the flowchart may represent a module, a segment, or a portion of program code, which includes one or more instructions executable by one or more processors for implementing specific logical functions or steps in the process. The program code may be stored on any type of computer readable medium, for example, such as a storage device including a disk or hard drive.

In addition, each block may represent circuitry that is wired to perform the specific logical functions in the process. Alternative implementations are included within the scope of the example implementations of the present application in which functions may be executed out of order from that shown or discussed, including substantially concurrent or in reverse order, depending on the functionality involved, as would be understood by those reasonably skilled in the art.

At block 202, method 200 involves receiving patient data for at least one patient at a centralized server system associated with at least one of a clinician device or a patient device, where the patient data includes historical health information. The centralized server can also receive patient data for a plurality of patients. As set forth above, the centralized server can be associated with one or both of the clinician device or the patient device. The clinician device and/or the patient device can transfer data back and forth via the centralized server. Alternatively, the clinician device or the patient device could be associated with different servers. For example, the clinician device can be associated with the centralized server and the patient device can be associated with a second server, or vice versa.

The method can include that the patient data for the at least one patient is gathered by a plurality of wearable devices worn by the patient. For example, wearable devices can include blood pressure monitors, heart rate monitors, step trackers, sphygmomanometers, blood sugar monitors, and smart watches. Additionally, the patient data can be gathered from auditory devices, patient questionnaires, existing healthcare data, clinician diagnoses, and clinician notes. The patient data can include disease diagnoses of the patient. For example, migraine diagnoses, broken bones, sprains, and past surgeries. Patient data can further include family health information.

As used herein, the patient data can refer to any data related to a patient. For example, patient data can include bio-signals, health state information, and questionary information of the user. The bio-signal may include at least one of an electrocardiogram signal, an ultrasonic signal, EDA (electrodermal activity), a body temperature, and a body fat percentage. Further, the questionary information may include at least one of information of a habit, a syndrome, a stature, a weight, and a waist measure of the user. The health state information may include information of at least one of medication usage data, whether a hospital visit is required, a stress/fatigue degree, a health age, a skin state, an obesity degree, a body portion balance degree, a heart health degree, a digestive organ health degree, and a respiratory organ health degree.

Patient data is not necessarily limited to health information. In a non-limiting example, patient data can include information of at least one of demographic characteristics, gender, race, lifestyle, diet, location, religion, mood, body type, physical appearance of the patient, and the patient's clothing size. As previously mentioned, the patient data can also include historical health information. The historical health information is data related to the health of a patient and can be compiled over a temporal period. The historical health information can be considered an archive of patient health to display on the patient avatar. For example, the historical health information can include but is not limited to medical records, health data, diagnostic information, acute and chronic illnesses, infection, and blood chemistry gathered from the patient's birth up to and including the present time. Alternatively, the historical health information can include data gathered sometime after the patient's birth up to and including the present time. Alternatively still, the historical health information can include data gathered sometime after the patient's birth up to a time before the present time.

In an example embodiment, the historical health information also includes at least one disease symptom, disease trigger, or disease factor. As previously discussed, disease symptoms can include an observable manifestation of a particular disease or disorder. In non-limiting examples, where the disease is migraine, the disease symptom can be a migraine headache, neck ache, head pain, sensitivity to light, sensitivity to sound, nausea, dizziness, and aura. Disease symptoms can further include body aches, stomach aches, and rashes. The historical health information for disease symptoms can further include (i) when the disease symptom occurred; (ii) how long the disease symptom lasted; (iii) the intensity and/or severity of the disease symptom; (iv) the location of the disease symptom; and/or (v) whether the disease symptom was accompanied by other related symptoms, and if so, the time, duration, intensity/severity of the accompanying symptoms.

In an example embodiment, the historical health information also includes therapeutic intervention data. Therapeutic intervention data can include actions or steps taken to reduce the effect of the disease symptoms on the patient. In non-limiting examples, therapeutic intervention data can include medication usage, exercise, yoga, diet, vagal nerve stimulators, sleep, and meditation. The therapeutic intervention data can further include (i) when the therapeutic intervention occurred; (ii) the type of therapeutic intervention data: (iii) and the length of time of the therapeutic intervention.

At block 204, method 200 involves generating the patient avatar, based on the patient data for the at least one patient, where the patient avatar includes a two-dimensional or three-dimensional graphical representation of the at least one patient, and where the historical health information is visually displayed as physical characteristics of the patient avatar. In an example embodiment, the two-dimension or three-dimensional patient avatar can be generated using avatar generation applications and tools that are generally known in the art, including but not limited to Pixologic ZBrush and Autodesk Maya. Other example avatar generation applications and tools include, but are not limited to, Ready Player Me, Avatar Maker, Union Avatars, Soul Machines, Unreal Engine, and In3D. Other methods of generating two-dimensional and three-dimensional avatars that are known to those of ordinary skill in the art are also contemplated herein. In an example embodiment, the patient avatar can resemble the at least one patient. The at least one patient can enter physical characteristics that can be used to generate the avatar. In an additional embodiment, the physical characteristics of the avatar can be generated based on photographic image data of the at least one patient. The patient avatar can also be generated based on the patient data previously mentioned such as demographic characteristics, gender, race, lifestyle, diet, location, religion, mood, body type, physical appearance of the patient, and the patient's clothing size.

The method can include generating the patient avatar at the centralized server, the clinician device, and/or the patient device. For example, the patient avatar can be generated at the clinician device and sent via the centralized server to the patient device. The patient device can also generate an updated version of the patient avatar and send it to the clinician device via the centralized server. Alternatively, the patient avatar can be generated at the patient device and sent via the centralized server to the clinician device. The clinician device can also generate an updated version of the patient avatar and send it to the patient device via the centralized server. Alternatively still, the patient avatar can be generated and updated at the centralized server and sent to the clinician device and/or the patient device.

In an example embodiment, the patient avatar includes a two-dimensional or three-dimensional graphical representation of at least one patient. The two-dimensional or three-dimensional graphical representation of at least one patient can allow a patient and/or a clinician to easily view the patient avatar in detail, including specific portions of the patient avatar. In an example embodiment, the historical health information is visually displayed as physical characteristics of the patient avatar. As set forth above, the historical health information can include at least one disease symptom. In an example embodiment, the physical characteristics of the patient avatar can be based on the at least one disease symptom of the at least one patient. In other words, the disease symptoms experienced by the patient can be visually reflected on the patient avatar. For example, the physical characteristics of the patient avatar can include an appearance of a symptomatic patient body part.

In an example embodiment, when the avatar is generated, the disease symptoms experienced by the at least one patient can be mapped to the appearance of body parts on the patient avatar. For example, a headache can be mapped to the appearance of the patient avatar's head, neck aches can be mapped to the appearance of the patient avatar's neck, stomach aches can be mapped to the appearance of the patient avatar's stomach, sensitivity to light can be mapped to the appearance of the patient avatar's eyes, and sensitivity to sound can be mapped to the appearance of the patient avatar's ears. Alternatively, when the patient has not experienced any disease symptoms, the patient may be calm, and the appearance of the patient avatar body parts may not have any mapped symptoms.

At block 206, method 200 involves displaying the patient avatar on a graphical user interface (GUI) associated with at least one of the clinician device or the patient device. The clinician device or the patient device can include screens to display the patient avatar GUI. The method further includes at block 206, that displaying the patient avatar includes displaying most recent patient data based on the historical health information. In an example embodiment, most recent patient data can be all of the historical health data that was gathered on the day closest to the day the patient avatar is displayed. For example, if the most recent historical health data is from a week before the patient avatar is being viewed on the clinician device and/or the patient device, the patient avatar can display the historical health data from a week before the avatar is viewed. Alternatively, if the most recent historical health data is from the day that the patient avatar is being viewed on the clinician device and/or the patient device, the patient avatar can display the historical health data from the day that the patient avatar is being viewed.

In an example embodiment, the GUI could be generated using programs of the Adobe Cloud package. However, generating the GUI is not limited to using the Adobe Cloud package. Other methods of generating GUIs known by persons of ordinary skill in the art are also contemplated herein.

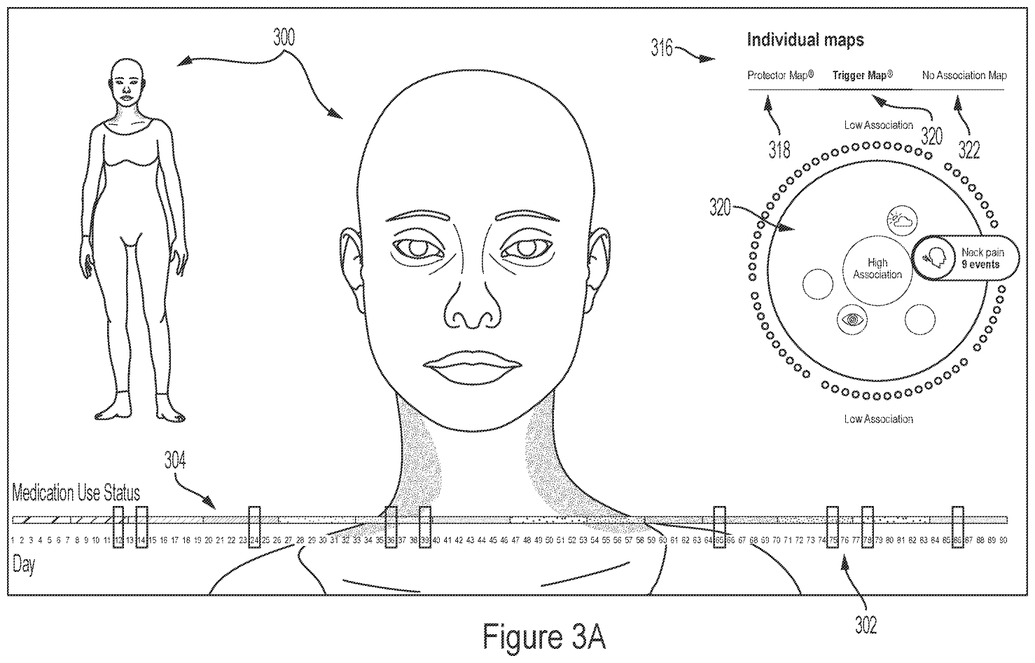

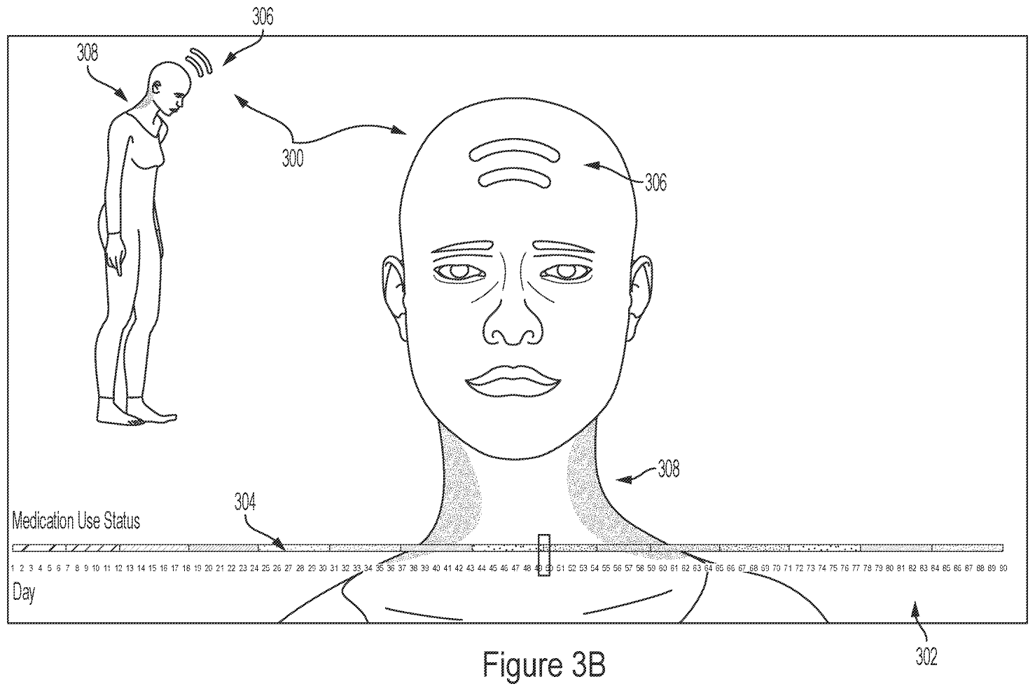

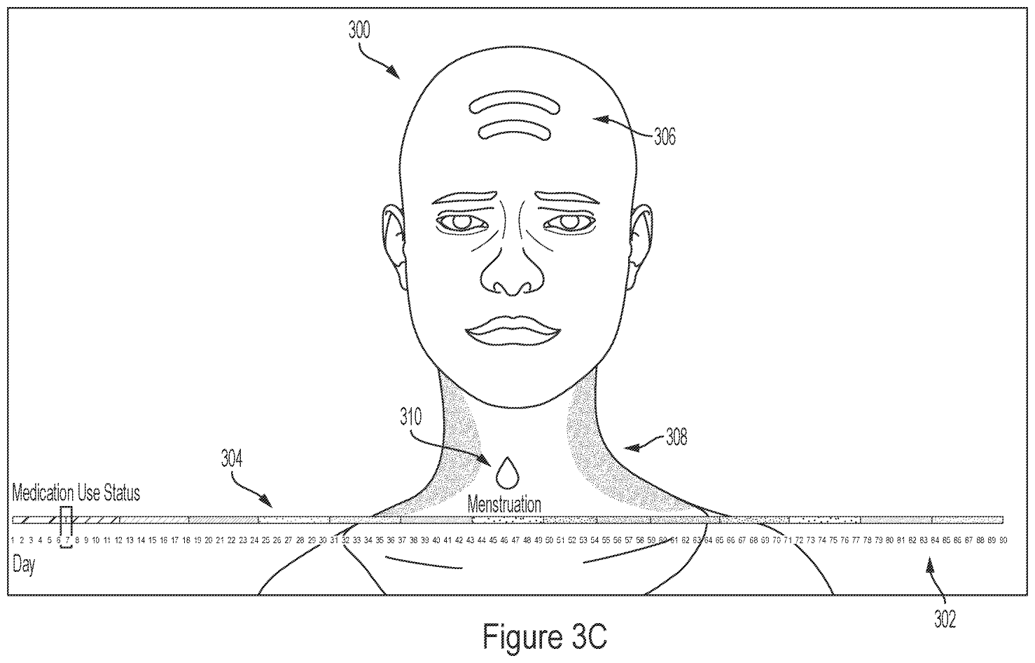

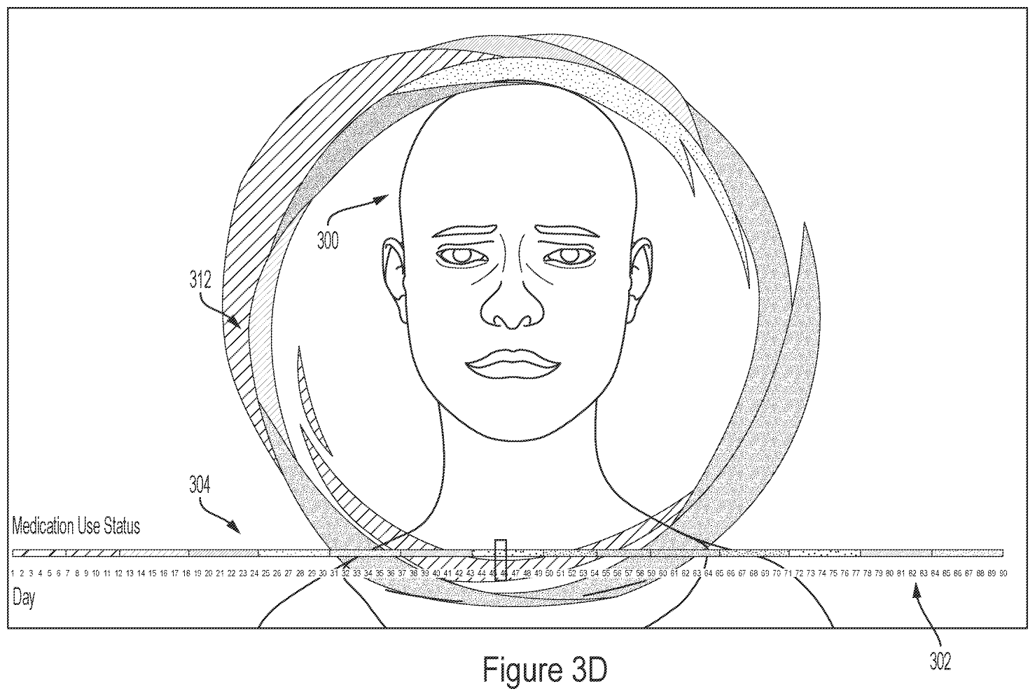

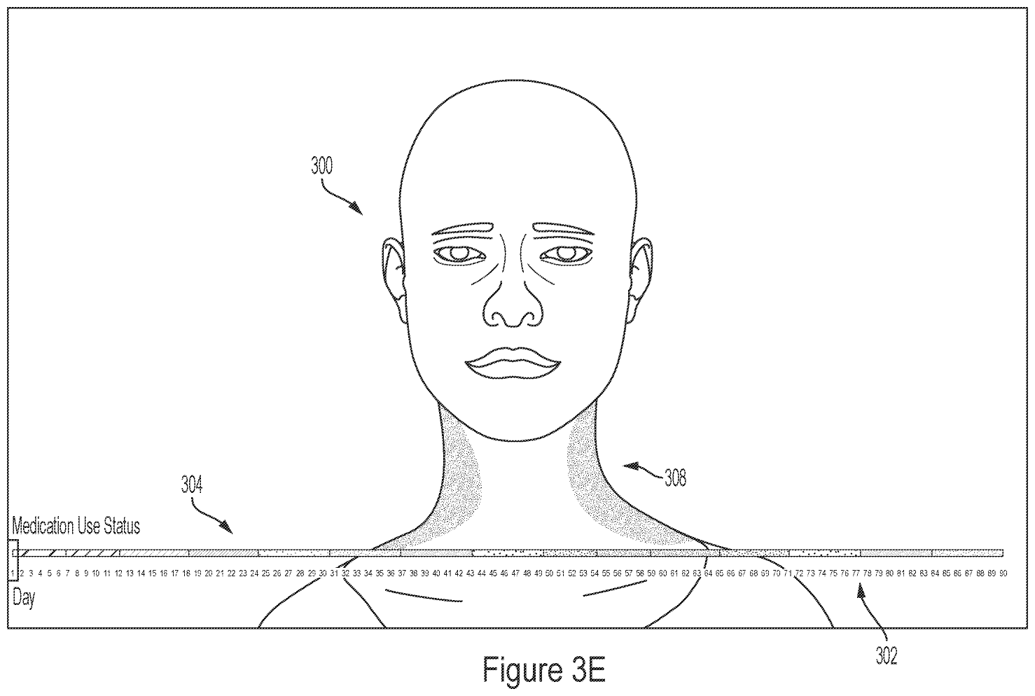

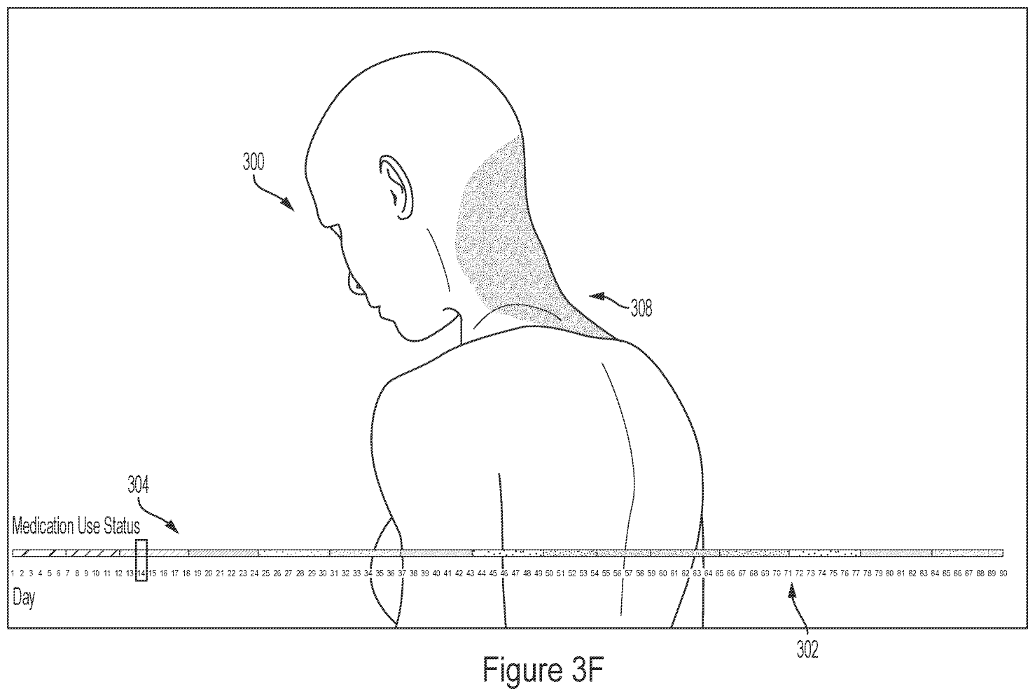

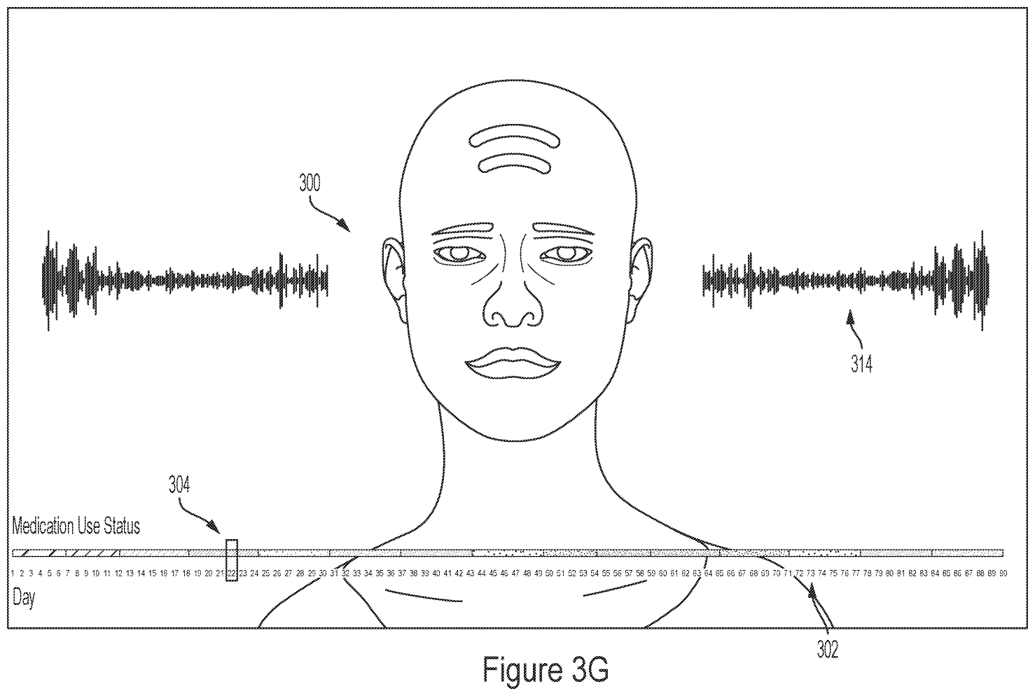

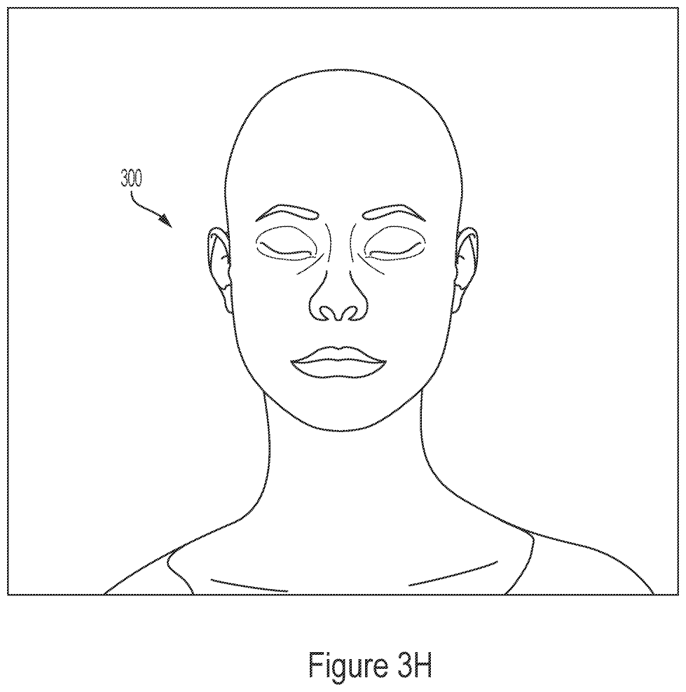

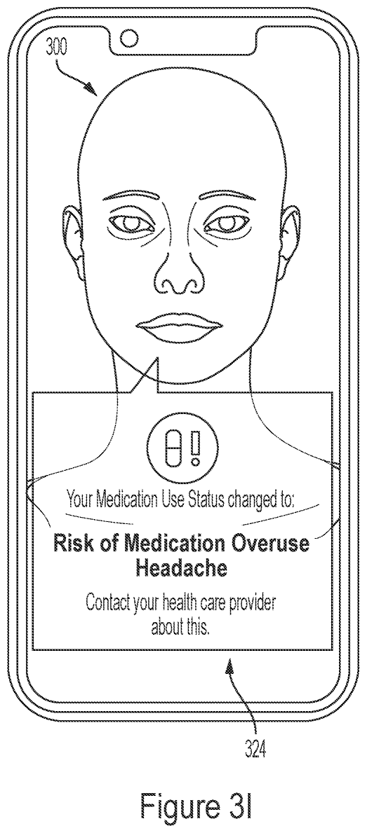

FIG. 3A illustrates the patient avatar 300 displayed on the clinician device and/or the patient device. As illustrated, the avatar can be displayed as a full body, or zoomed into a specific portion of the patient avatar body. In an example embodiment, the GUI can include a timeline 302 to indicate what day the patient avatar is displaying historic health information from. The GUI can also include a block 304 to indicate medication use status for a predetermined disease condition on each of the days in the timeline 302. The block 304 can indicate based on color, whether the patient's medication use is healthy or bordering on overuse. For example, shades of green can represent that there is no chance of medication overuse on the related day while shades of red can represent that there is a chance of medication overuse on the related day. Alternatively, the block can use shading, color gradients, gray scale gradients, color tones, patterns, symbols, or shapes to indicate whether the patient's medication use is healthy or bordering on overuse. For example, the darker the color becomes, or the denser the pattern becomes can indicate the chance of medication overuse increasing. The predetermine disease condition can be selected to determine, based on guidelines, whether there is a risk of medication overuse. For example, the current ICHD-3 Guidelines can be used to determine whether a patient is at risk of medication overuse related to migraine or headache. Other guidelines may apply to different conditions.

The GUI can also include a plurality of individual maps 316. The plurality of individual maps 316 can include at least a protector map 318, a trigger map 320, or a no association map 322. As illustrated, the trigger map 320 can include a plurality of disease triggers and disease symptoms. When a disease trigger or disease symptom is selected on the trigger map 320, the days which correspond to the selected disease trigger or disease symptom can be highlighted on the timeline 302. Some aspects of generating the plurality of individual maps are described in PCT Application PCT/US2015/043945 filed on Aug. 6, 2015, the contents of which are incorporated herein by reference.

In an example embodiment, as previously mentioned, the physical characteristics of the patient avatar can be based on the at least one disease symptom of the at least one patient. When displayed, the appearance of a symptomatic body part can be altered. The appearance can include: a size of the symptomatic patient body part, a color of the symptomatic patient body part, a shade of the symptomatic patient body part, a texture of the symptomatic patient body part, and an icon or symbol displayed on the symptomatic body part. For example, sensitivity to sound can be displayed on the patient avatar as enlarged ears, solid red ears, shaded red ears, blurry ears, ears of a different texture, ears with a pattern on then, ears with a symbol on them, and/or sound waves bombarding the ears. Similar examples can apply to other patient avatar body parts as well.

FIGS. 3B-3G illustrate, but are not limited to, a plurality of symbols and changes in the appearance of symptomatic body parts. Other changes and alerts are also possible. In an example embodiment, FIG. 3B illustrates that on day 49 of the historical health information, the patient was experiencing a migraine 306 and neck pain 308. The migraine 306 and neck pain 308 can be illustrated on both a zoomed in and full body display of the avatar 300. The migraine 306 is displayed as a plurality of curved lines on or above the patient avatar 300 forehead. The migraine can also be displayed by red shading on the patient forehead. Alternatively, the migraine can be indicated by shading, color gradients, gray scale gradients, color tones, patterns, symbols, or other shapes on the head. The neck pain 308 is displayed as a red shading on the neck of the patient avatar 300 in the location of the pain. Alternatively, the neck pain can be indicated by shading, color gradients, gray scale gradients, color tones, patterns, symbols, or shapes on the neck. FIG. 3C illustrates that on day 6 of the historical health information, the patient was experiencing migraine 306, neck pain 308, and menstruation 310. Menstruation 310 is displayed on the GUI as a red drop symbol with a label. Alternatively, menstruation can be indicated by words, or other symbols, or shapes. FIG. 3D illustrates that on day 45 of the historical health information, the patient was experiencing aura 312. Aura 312 can be defined as visual distortion. Aura 312 is displayed on the patient avatar 300 as an ethereal swirl around the patient avatar's 300 head. Alternatively, the aura can be displayed using shading, color gradients, gray scale gradients, color tones, patterns, symbols, or shapes. The change in the indicator can also show the intensity of the aura. For example, the darker the color becomes, or the denser the pattern becomes can indicate a more intense aura. FIG. 3E illustrates that on day 1 of the historical health information, the patient was experiencing neck pain 308. The neck pain 308 can be displayed as a red shading on the sides of the neck of the patient avatar 300. Alternatively, the neck pain can be indicated by color gradients, gray scale gradients, color tones, patterns, symbols, or shapes. FIG. 3F illustrates that on day 14 of the historical health information, the patient was also experiencing neck pain 308 but in a different location than in FIG. 3E. The neck pain 308 is displayed as a red shading on the back of the neck of the patient avatar 300. Alternatively, the neck pain can be indicated by color gradients, gray scale gradients, color tones, patterns, symbols, or shapes. FIG. 3G illustrates that on day 22 of the historical health information, the patient was experiencing migraine 306, and sensitivity to noise 314. The sensitivity to sound 314 is displayed as noise waves entering each of the ears of the patient avatar 300. The noise waves can be a plurality of colors. Alternatively, the noise waves can be color gradients, gray scale gradients, color tones, patterns, or other shapes. In an additional embodiment, sensitivity to noise can also include shading the ears of the patient avatar red. Alternatively, the ears of the patient avatar could include color gradients, gray scale gradients, color tones, patterns, symbols, or shapes to indicate sensitivity to noise. FIG. 3H illustrates the patient avatar 300 at rest, with no patient data to display.

Method 200 can further include determining a trend in historical health information. In an example embodiment, the trend can be defined by health guidelines to be clinically significant. For example, “pill popping” frequency that is defined by National Health Guidelines to be clinically significant. Here, clinically significant can mean that the medication use will present a health risk. Additional examples of trends defined by health guidelines to be clinically significant include HbA₁C trends towards meeting diagnosis of diabetes defined by the World Health Organization, and blood pressure trends defined by the American College of Cardiology, which indicate need for lifestyle and therapeutic intervention. Trends can also be marked by a change in the frequency, severity, and/or duration of a patient disease symptom, action, or environmental factor. The trend, or change in trend, can be determined through the historical health information. Once the trend is determined, method 200 can include displaying, on the graphical user interface associated with at least one of the clinician device or the patient device, an alert of the trend. Displaying the alert can include at least one of an in-avatar alert and an ex-avatar alert, meaning either or both the in-avatar alert and the ex-avatar alert could be displayed. In an example embodiment, the in-avatar alert includes changing avatar appearance changing at least one of a size of a body part, a color of a body part, a shade of a body part, or a texture of a body part. The in-avatar alerts can be used to alert the clinician and/or the patient of a status or a trend of an aspect of the patient's body. The ex-avatar alert can include at least one of a symbol on the outside the patient avatar and a text box. Ex-avatar alerts can be used to alert the clinician and/or the patient about something external related to a disease factor such as use of medication. FIGS. 3B, 3C, 3E, and 3F illustrate examples of in-avatar alerts. However, in-avatar alerts are not limited to the alerts in FIGS. 3B, 3C, 3E, and 3F. FIGS. 3B, 3C, and 3G illustrate examples of ex-avatar alerts. FIG. 3I illustrates a further example of an ex-avatar alert on the GUI. The ex-avatar alert can be a text box 324, which could be displayed in a variety of colors. In an example embodiment, the text box 324 can be a dialog bubble from the patient avatar 300. Although ex-avatar alerts are illustrated in FIGS. 3B, 3C, 3D 3G, and 3I, it should be understood that ex-avatar alerts are not limited to the figures.

In an example embodiment, the alert can also take an audible form. When the alert of the trend is triggered, an alert sound could be played from at least one of the patient device, the clinician device, or an external speaker. The external speaker can include at least one of a smart speaker, a surround speaker, a sound bar, a portable speaker, a Bluetooth speaker, or headphones. The type of alert and the severity of the alert can be indicated by the sound. For example, the volume, tone, pattern, or other aspect of the alert sound can increase or change as the severity or urgency of the alert increases. In a non-limiting example, the different trends can also be marked by different sounds.

In an example embodiment, the body movement of the patient avatar can also be used to alert the clinician and/or the patient. Depending on the alert level, movement of the patient avatar could have a hierarchy: for example, first the patient avatar taps its toes, then waves its arms, then jumps up and down, and then starts yelling.

In a further example embodiment, the alerts can be customized by the clinician and/or the patient. The clinician and/or the patient could decide what type of alerts to display (e.g., in-avatar, ex-avatar, avatar movement) and the frequency of alerts.

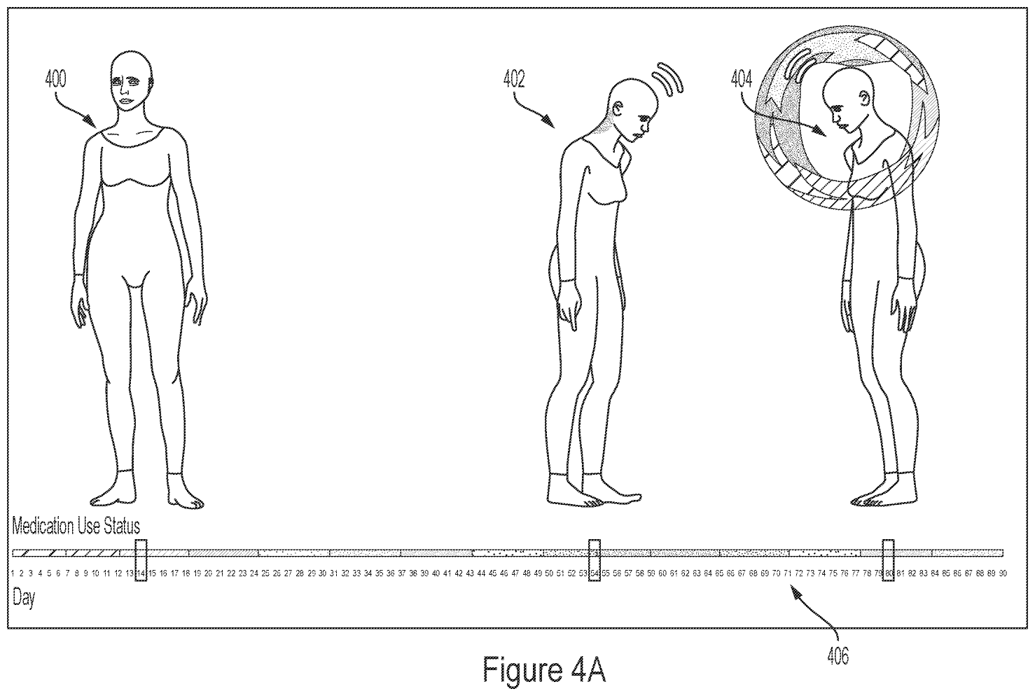

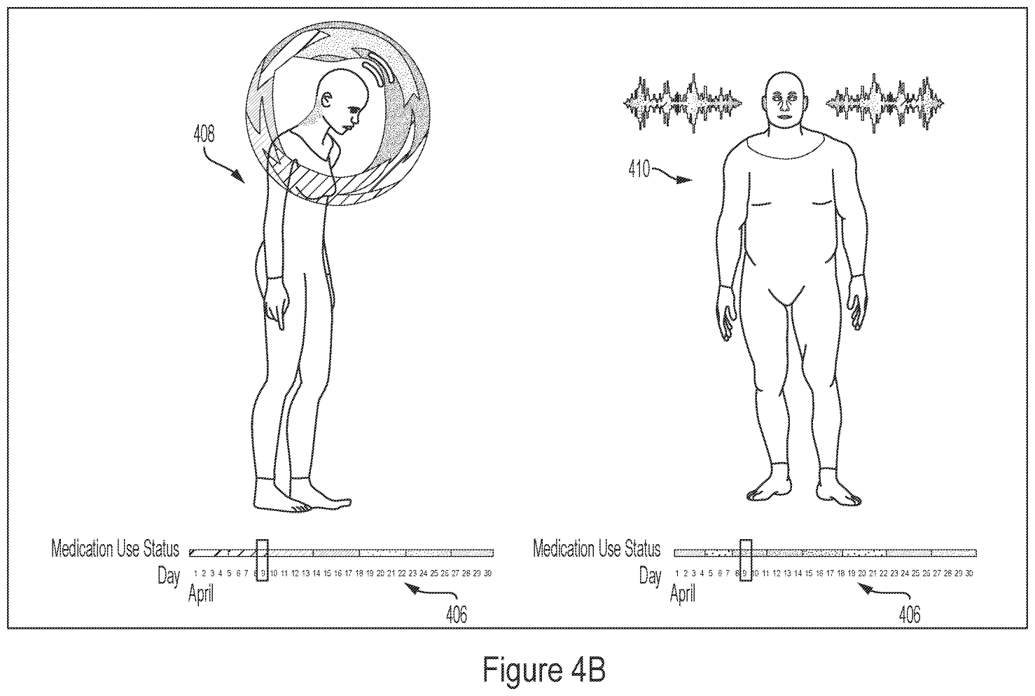

These methods can also include displaying the patient avatar and at least one additional avatar on the graphical user interface associated with at least one of the clinician device or the patient device. The patient avatar and the additional avatar can be displayed side-by-side on the graphical user interface. FIG. 4A illustrates an example embodiment of the patient avatar displayed with additional avatars as a way to compare the avatars. In the Figure, the patient avatars are for one patient, but displaying historic health information from different times. In an example embodiment, the patient avatar can be generated based on the patient data from a first time period in the historical health information and the additional avatar can be generated based on the patient data for the at least one patient from a second time period in the historical health information. For example, FIG. 4A includes a GUI displaying comparisons of the patient avatar at different days. The GUI can display a first patient avatar 400 which is displaying historic health information from day 14 according to the timeline 406, second patient avatar 402 which is displaying historic health information from day 54 according to the timeline 406, and third patient avatar 404 which is displaying historic health information from day 80 according to the timeline 406. In an additional embodiment, the avatars can display averages of the historical health data (i.e., first patient avatar displays an average of the historic health information from days 7 to 14, while second patient avatar displays an average of the historic health information from days 60 to 67) next to each other in order to compare the avatars.

In an example embodiment, the additional avatar can be generated based on additional patient data for at least one additional patient. In a non-limiting example, the at least one additional patient is not the at least one patient. The additional avatar can include a two-dimensional or three-dimensional graphical representation of the at least one additional patient, and the additional historical health information can be visually displayed as physical characteristics of the additional avatar. This can allow two patient avatars, from two different patients to be compared to each other. FIG. 4B illustrates an example embodiment of the patient avatar displayed with one additional avatar that is not the patient avatar as a way to compare the avatars on the same day. The GUI can display a patient avatar 408 which is displaying historic health information from day 9 according to the timelines 406, and an additional patient avatar 410 which is displaying historic health information from day 9 according to the timeline 406. The patient avatar and the additional patient avatar can be grouped together based on a common feature or disease symptom that may cluster them. In an alternative embodiment, the additional patient avatar can be compared to the patient avatar in response to a request from the patient avatar.

The patient avatar and the additional patient avatar can display historical health information from the same day, from different days, from the same span of days, or from different spans of days. In other words, the patient avatar and the additional patient avatar can be generated from historical health information from one day, or an average of historical health information over a span of days.

At block 208, method 200 involves playing back a graphical representation of a temporal evolution of the patient avatar in response to receiving a playback command from at least one of the clinician device or the patient device. The temporal evolution of the patient avatar can include a change over time in historical health information of the at least one patient.

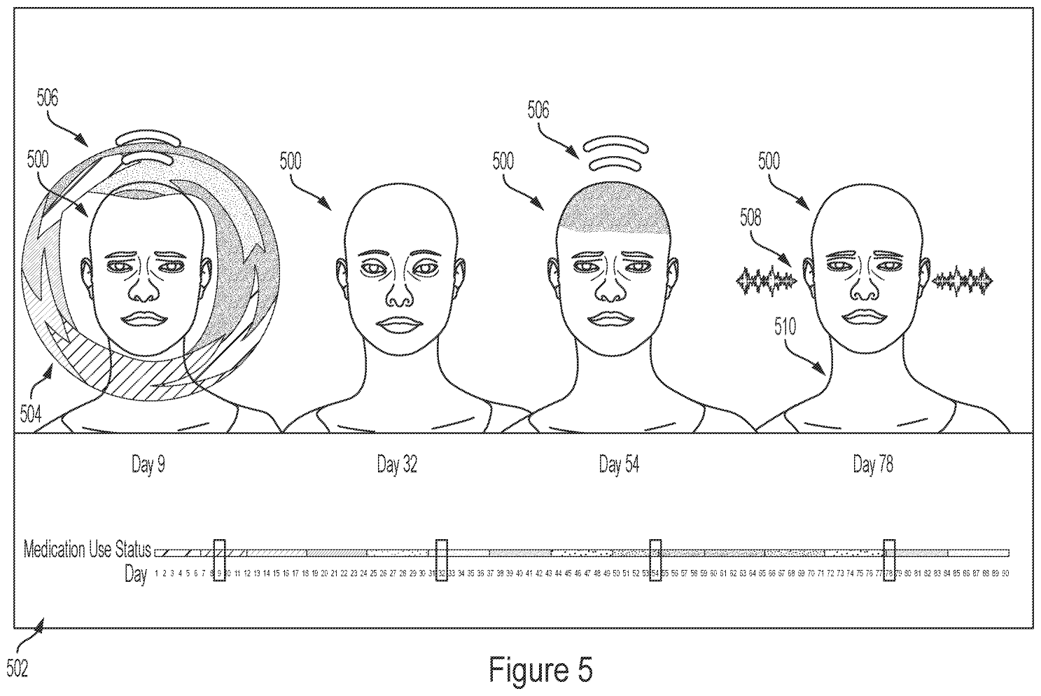

In an example embodiment, playing back the graphical representation of the temporal evolution of the patient avatar includes displaying changing disease symptoms of the patient. In other words, the patient avatar and the patient avatar's characteristics can be animated so that from a first day to a subsequent day, the patient avatar alerts and characteristics can change. FIG. 5 includes a GUI displaying a patient avatar 500 as the historic health information that the avatar displays evolves over a temporal period of 90 days. As illustrated, according to the timeline 502, on day 9 the patient avatar 500 displays aura 504 and migraine 506. As the graphical representation of the patient avatar changes over the temporal period, on day 32 the patient avatar does not have any patient data to display. FIG. 5 illustrates that on day 54 the patient avatar displays migraine headache 506, and that on day 78 the patient avatar displays neck ache 508 and sensitivity to noise 510. FIG. 5 also illustrates that the size of the symbol can represent the severity of the disease symptom. For example, the migraine symbol on day 9 is larger than the migraine symbol on day 54, indicating that the migraine on day 9 could have been more severe. In an additional embodiment, the opaqueness of the shading can also indicate severity. The more opaque the shading, the more severe the disease symptom may be. Other symbols can also be displayed to illustrate the severity of the disease symptom.

The playback feature described herein can also be used when comparing avatars. For example, the patient avatar can display a playback of the historical health data from days one through seven, while the additional patient avatar can display a playback of the historical health data from days 21 to 28. In an additional embodiment the playback feature can also be used to compare the patient avatar to at least one additional avatar that is not based on the historical health information from the patient. For example, the patient avatar can display a playback of the historical health data from days one through seven, and the additional patient avatar that is not based on the historical health information from the patient can also display a playback of the historical health data from days one through seven.

In an example embodiment, the clinician and/or the patient may be able to adjust the playback speed of the graphical representation of a temporal evolution of the patient avatar. Fast playback speeds may be beneficial to quickly grasp the temporal evolution of the patient avatar, while slower playback speeds may be beneficial to gather details of the historical health information from the patient avatar. In another example embodiment, the timeline 502 can be a sliding bar that can be used to jump to specific days in the historical health information. From those days, more detailed historical health information can be displayed.

In an example embodiment, the method further includes that playing back the graphical representation of the temporal evolution of the patient avatar includes displaying changes in historical health data from a previous clinician appointment to a current clinician appointment. For example, the clinician may add to the historical health information in the previous meeting and may add additional historical health information in a current meeting. In this embodiment, a clinician and/or a patient can view the change in historical health information from between the two appointments.

In an additional embodiment, the patient avatar can also be used to obtain specific health information. An example embodiment can include prompting the patient avatar with a historical health information question, such as “when was the last migraine the patient experienced.” The method can include querying through the patient data to find and answer to the question. A user may then receive an answer to the historical health information question, based on the historical health information.

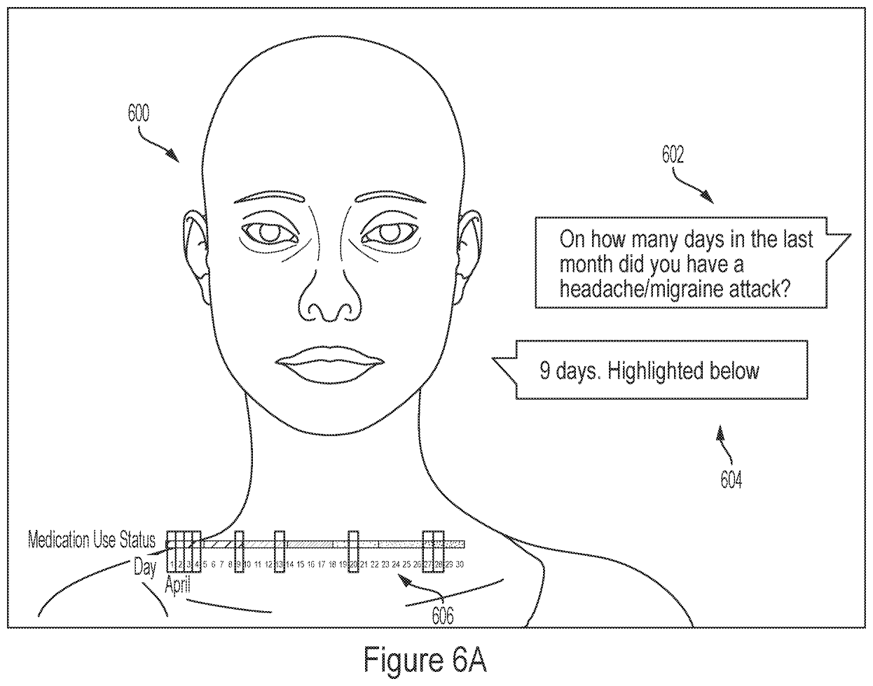

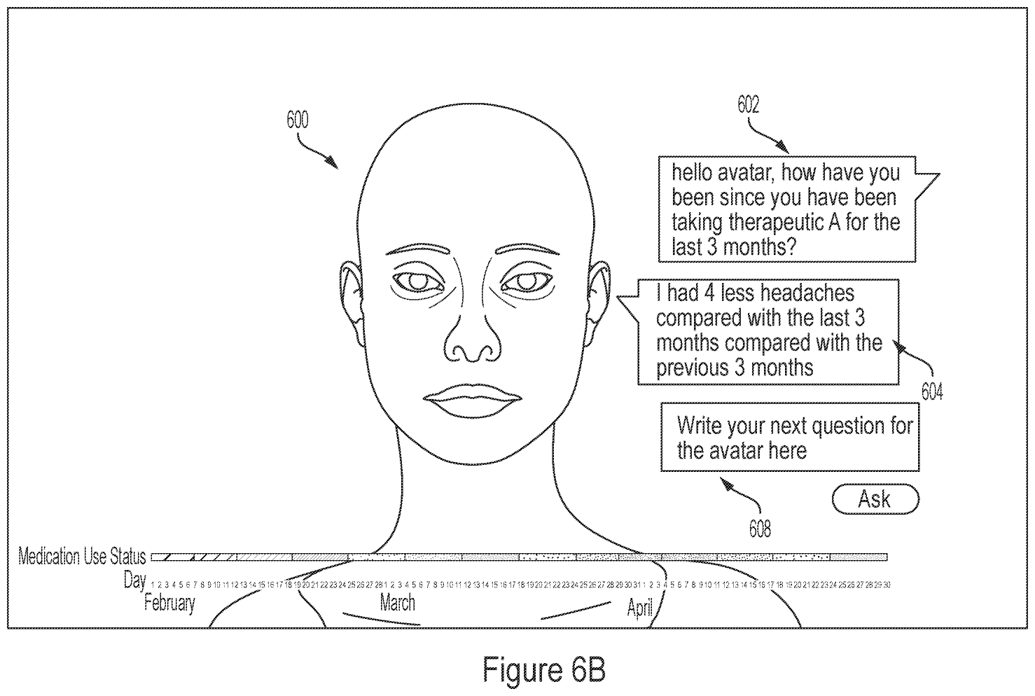

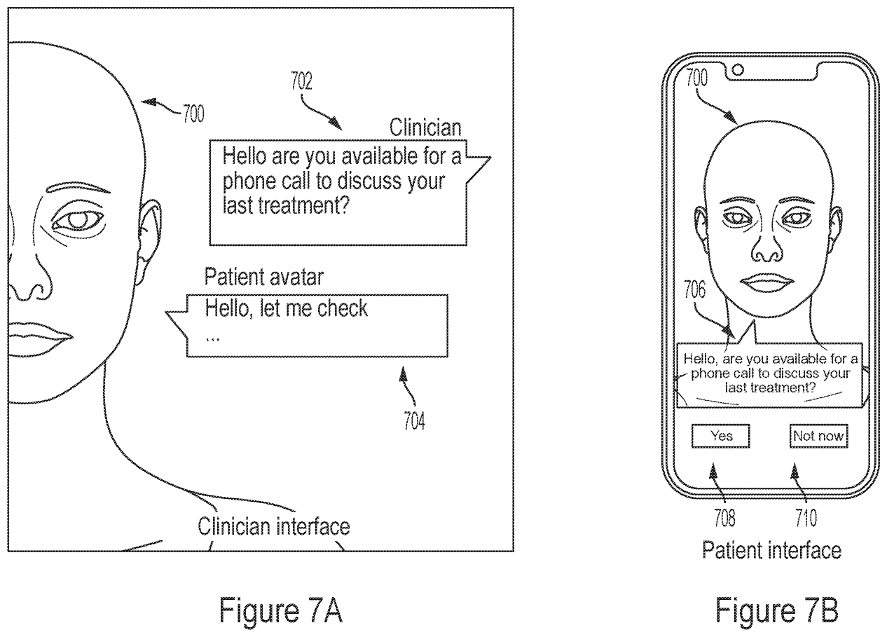

FIGS. 6A and 6B illustrate an example embodiment of the patient and/or the clinician asking the patient avatar a specific health question. The avatar can consult the patient data and provide an answer. For example, in FIG. 7A the clinician and/or the patient asks the patient avatar 600 how many days in the last month the patient had a headache/migraine attack, which appears in a question box 602. The patient avatar provides an answer in an answer box 604 and also provides an indication of the days on the timeline 606 that are associated with the answer to the health question. In FIG. 6B the clinician and/or the patient asks the patient avatar 600 how the patient has been since taking a therapeutic for the last three months, as displayed in question box 602. The patient avatar provided the answer that the patient had 4 less headaches in the last three months compared with the previous three months in answer box 604. The patient and/or clinician can then write another question in the question box 608.

Example embodiments described herein can also include inputting a question command via the graphical user interface associated with the clinician device, and in response to receiving the question command, displaying the question command on the graphical user interface associated with the patient device. The question command entered by the clinician could be related to missing patient data, or a specific disease symptom. In an example embodiment the clinician can type the question into the GUI. The question could then be sent from the clinician device to the GUI of the patient device for the patient to view and answer. In an additional embodiment, the clinician could dictate and record a question command. The auditory question command could then be sent to the patient GUI for the patient to listen to. In an additional embodiment, the clinician could type the question into the GUI. The question could be sent to the patient device and converted to speech for the patient to listen to. The patient can respond to any of the previously described received questions by typing in a response or by recording a vocal response. The clinician device can receive the text and/or the vocal response, and in an example embodiment the text can be displayed on the GUI of the clinician device and/or convert the vocal response into text to display on the GUI of the clinician device.

The method can also include inputting a request for a real-time conversation via the graphical user interface associated with the clinician device. A real-time conversation could include a real-time texting conversation between the patient and the clinician or a call between the patient and the clinician. In response to receiving the request, the GUI can display the request for the real-time conversation on the graphical user interface associated with the patient device. The request can include at least an option to accept, deny, or schedule the real-time conversation. The patient can initiate the real-time conversation with the clinician upon accepting the real-time conversation. The patient can also accept the request for the real-time conversation and then schedule it for a later time.

FIGS. 7A and 7B illustrate an example embodiment of a request for a real-time conversation via the patient avatar GUI 700. The clinician can send a request 702 through the patient avatar GUI on the clinician device for a phone call. The patient avatar can give a temporary response 704 to the clinician while the patient avatar contacts the patient. The patient avatar GUI 700 can then send the patient an availability question 706 on the patient device. The patient can select yes 708 to accept the request, or not now 710 to deny the request.

As the patient avatars are archives of patient data, added security is beneficial when sending the avatars between devices. In an example embodiment, the patient avatar comprises a non-fungible token (NFT) to authenticate that the patient data associated with the patient avatar is accurate. In other words, the patient avatar can be a unique and irreplaceable digital representation of patient data making the patient avatar a unique, non-transferable identity to distinguish it from other tokens. The patient avatar can also be extensible to allow the patient data to be updated. For example, the patient avatar can be an NFT stored on the centralized server. The patient can input additional patient data via the GUI of the patient device which can also be an NFT. The two NFTs can be combined to update the patient avatar with accurate patient data. The same can be true for patient data added by the clinician.

In an example embodiment, private and public keys can be used when sending patient data to update the patient avatar, and when sending the patient avatar NFT. The clinician device can further include a clinician identity. The clinician identity can include a clinician private key used to decrypt received patient data that has been encrypted and a clinician public key used to encrypt the patient data to send. In the method described herein, receiving patient data can include receiving encrypted patient data by the clinician public key and decrypting the patient data by the clinician private key. The historical health information can also be transmitted from a patient identity. The patient identity can include a patient private key used to decrypt received patient data that has been encrypted and a patient public key used to encrypt the patient data to send.

An example embodiment can include sending the patient avatar from a patient public key to a recipient public key. The patient avatar can include all of the patient data which includes historical health information. The patient data in the avatar can be encrypted and sent from the patient public key to the recipient public key. The recipient public key can belong to at least one of another patient (i.e., a client that also has a patient avatar), a clinician, or a patient's friend. The recipient public key can also be associated with a recipient private key. In an example embodiment the recipient private key can be compared to a database of private keys. The database of private keys can be made up of a list of a plurality of clinician private keys and a plurality of client private keys. The clinician private keys can be associated with a clinician while the client private keys can be associated with anyone that is not a clinician. In some examples, each particular patient could have a specific client private key. Based on a determination that the recipient private key is a clinician private key; the entire patient avatar can be shared with the private key. Once the entire patient avatar is shared with the clinician private key, the clinician private key can be used to decrypt the patient avatar and access the entire patient avatar. The entire patient avatar can be shared with the clinician so that the clinician has full access to the patient's patient data including historical health information on which to base a diagnosis.

Alternatively, if it is determined that the recipient private key is not a clinician private key, a predetermined portion of the patient avatar can be shared with the recipient private key for the recipient private key to decrypt. For example, the patient may wish to share a portion of the patient avatar with a friend. The patient can select a portion of patient data which they prefer to share with friends versus patient data which they would prefer to keep private. Once it is determined that the recipient private key is not a clinician private key, only the selected portion of patient data will be shared with the recipient private key for the private key to decrypt.

Example embodiments described herein can further be used in applications other than the health field. In non-limiting examples, the patient avatar previously described can further be used in clothing applications to try on clothing virtually, and to socially interact with other avatars. The benefits of the personalized patient avatar extend past health care and into the future of social interaction.

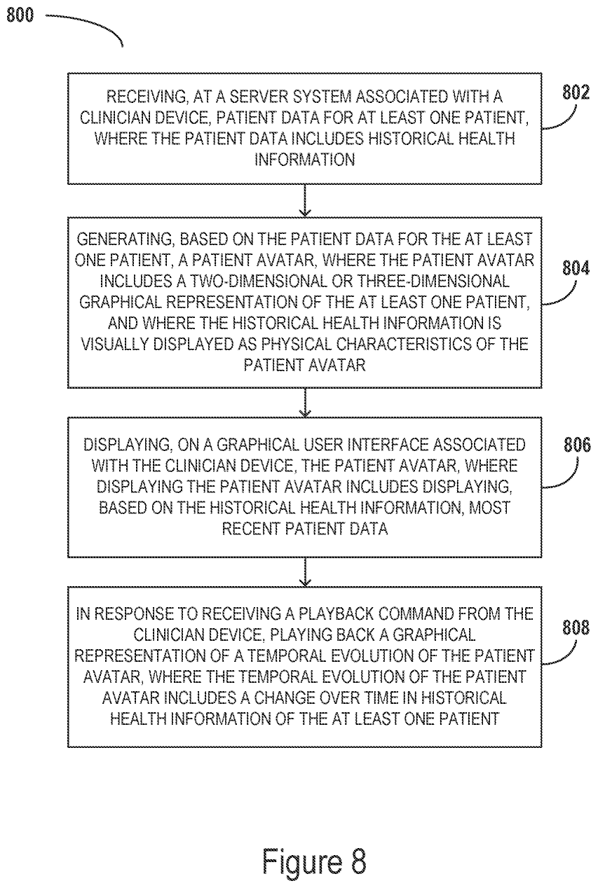

FIG. 8 is a flowchart of a method of interacting with a patient avatar by a clinician, according to one or more example embodiments. Method 800 can include the embodiments described above. Method 800 may include one or more operations, functions, or actions, as depicted by one or more of blocks 802, 804, 806, and 808 each of which may be carried out by any of the systems shown in prior figures, among other possible systems.

Those skilled in the art will understand that the flow charts described herein illustrate functionality and operation of certain implementations of the present disclosure. In this regard, each block of the flowchart may represent a module, a segment, or a portion of program code, which includes one or more instructions executable by one or more processors for implementing specific logical functions or steps in the process. The program code may be stored on any type of computer readable medium, for example, such as a storage device including a disk or hard drive.

In addition, each block may represent circuitry that is wired to perform the specific logical functions in the process. Alternative implementations are included within the scope of the example implementations of the present application in which functions may be executed out of order from that shown or discussed, including substantially concurrent or in reverse order, depending on the functionality involved, as would be understood by those reasonably skilled in the art. The embodiments described above can be performed in method 800 as described below. Without limitation, other embodiments could also be performed using method 800.

At block 802, method 800 involves receiving patient data for at least one patient at a server system associated with a clinician device, where the patient data includes historical health information. The clinician device can transfer data back and forth with the server system.

At block 804, method 800 involves generating the patient avatar, based on the patient data for the at least one patient. The patient avatar includes a two-dimensional or three-dimensional graphical representation of the at least one patient, and the historical health information is visually displayed as physical characteristics of the patient avatar. The method can include generating the patient avatar at the server system and/or the clinician device. For example, the patient avatar can be generated at the clinician device and sent to the server system. The clinician device can also receive the patient avatar from the server system.

At block 806, method 800 involves displaying the patient avatar on a graphical user interface associated with the clinician device. Displaying the patient avatar includes displaying most recent patient data based on the historical health information.

At block 808, method 800 involves playing back a graphical representation of a temporal evolution of the patient avatar in response to receiving a playback command from the clinician device. The temporal evolution of the patient avatar can include a change over time in historical health information of the at least one patient, as set forth above.

In an example embodiment, the GUI can be used by the clinician to input new patient data. The GUI can receive patient health information from the clinician. The patient health information can be input into the GUI by typing it in, or by voicing it to the GUI. The patient health information entered into the GUI can then be compared to the historical health information. If it is determined that the patient health information from the clinician contains new data, the patient avatar can be updated based on the new data.

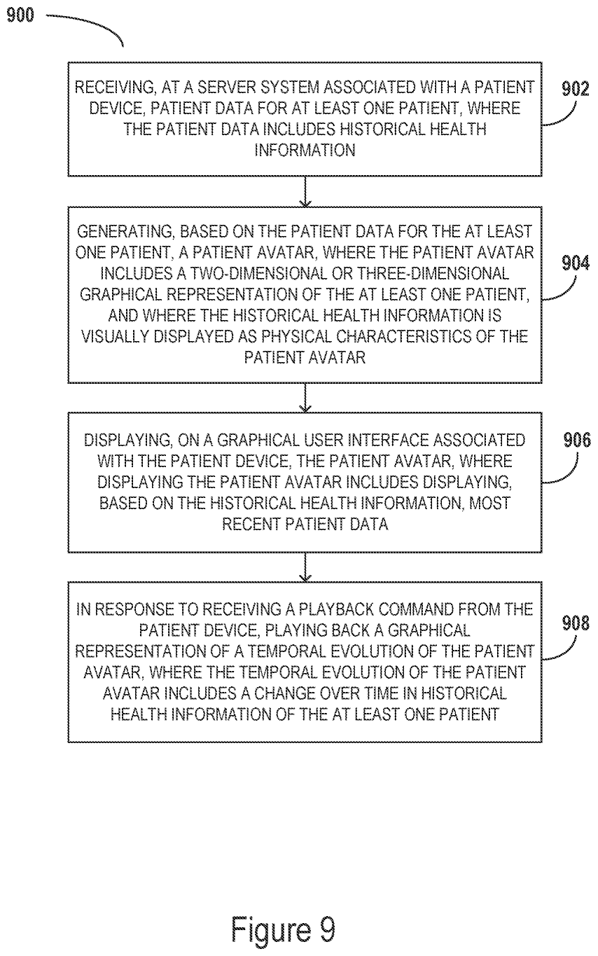

FIG. 9 is a flowchart of a method of generating a patient avatar, according to one or more example embodiments. The patient can interact with the patient avatar. Method 900 can include the embodiments described above. Method 900 can include one or more operations, functions, or actions, as depicted by one or more of blocks 902, 904, 906, and 908 each of which can be carried out by any of the systems shown in prior figures, among other possible systems.

Those skilled in the art will understand that the flow charts described herein illustrate functionality and operation of certain implementations of the present disclosure. In this regard, each block of the flowchart may represent a module, a segment, or a portion of program code, which includes one or more instructions executable by one or more processors for implementing specific logical functions or steps in the process. The program code may be stored on any type of computer readable medium, for example, such as a storage device including a disk or hard drive.

In addition, each block may represent circuitry that is wired to perform the specific logical functions in the process. Alternative implementations are included within the scope of the example implementations of the present application in which functions may be executed out of order from that shown or discussed, including substantially concurrent or in reverse order, depending on the functionality involved, as would be understood by those reasonably skilled in the art. The embodiments described above can be performed in method 900 as described below. Without limitation, other embodiments could also be performed using method 900.

At block 902, method 900 involves receiving patient data for at least one patient at a server system associated with a patient device. The patient data can include historical health information. The historical health information can include patient information as previously described and can also be gathered as previously described.

At block 904, method 900 involves generating the patient avatar based on the patient data for the at least one patient. The patient avatar includes a two-dimensional or three-dimensional graphical representation of the at least one patient, and the historical health information is visually displayed as physical characteristics of the patient avatar. These methods can include generating the patient avatar at the server system and/or the patient device. For example, the patient avatar can be generated at the patient device and sent to the server system. The patient device can also receive the patient avatar from the server system.

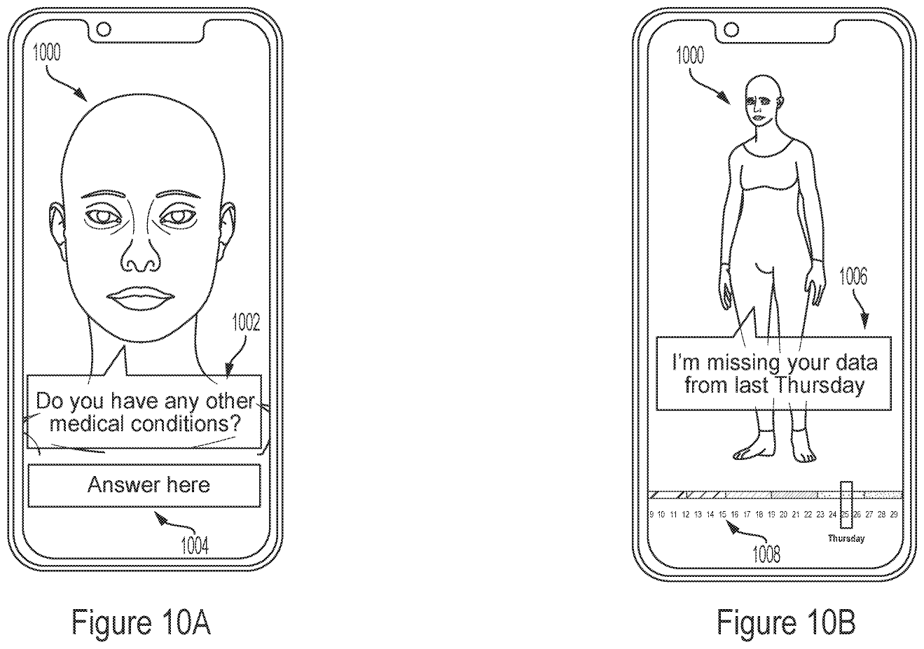

At block 906, method 900 involves displaying the patient avatar on a graphical user interface associated with the patient device. Displaying the patient avatar includes displaying most recent patient data based on the historical health information. In an example embodiment, the historical health information can include answers to personality profile questions. The answers to the personality profile questions can be gathered by prompting the patient to answer them via the GUI. FIG. 10A illustrates a prompt for the user to answer a question, which can be included in the personality profile questions. In FIG. 10A, the patient avatar 1000 asks a question which appears as a text box 1002 on the GUI. An “Answer here” box 1004 is positioned below the question for the patient to type the answer into. In an additional embodiment, the patient can speak the answers and the patient device could record the answers to the question or convert the audio into text.

In an example embodiment, the GUI can also prompt the patient to enter missing patient data. FIG. 10B illustrates an informational prompt 1006 for the user to inform them that data is missing from a specific day. In response to receiving the prompt, the patient can use the timeline 1008 to go to the day with missing data and enter the missing data. The missing data can be entered by typing in it, by voicing it, or by interacting with the patient avatar.

FIG. 1 IA illustrates a flow chart 1100 related to entering data by interacting with the patient avatar. Block 1102 includes activating the patient avatar via the GUI. The patient avatar can be activated by touching a screen of the patient device on which the GUI is displayed, or by voicing at least one specific word. Block 1104 includes zooming in on a symptomatic body part via the GUI. Block 1106 includes selecting the symptomatic body part on the patient avatar via the GUI displayed on the screen of the patient device. The flow chart then splits into at least two different routes. Block 1108a includes displaying a prompt to enter symptom data for the symptomatic body part. Block 1110a includes entering the symptom data for the symptomatic body part. Following the at least one other route of the flow chart, block 1108b includes displaying a submenu for the symptomatic body part. The submenu can include a plurality of disease symptoms for the symptomatic body part. For example, the symptomatic body part could include at least two disease symptoms associated with it that can be associated with different symptom data. Block 1108c includes selecting at least one option from the submenu. In a non-limiting example, options from the submenu can include at least two disease symptoms associated with the symptomatic body part. Block 1110b can include entering the symptom data for the at least one option from the submenu. Block 112 includes updating the patient avatar based on the symptom data entered in at least block 1110a and 1110b.

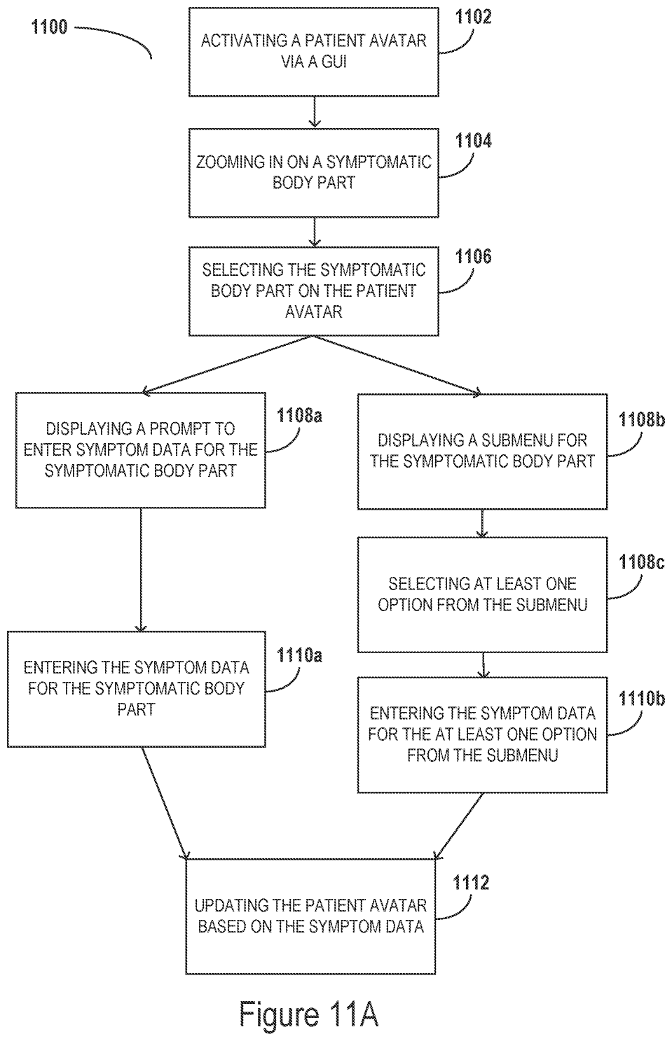

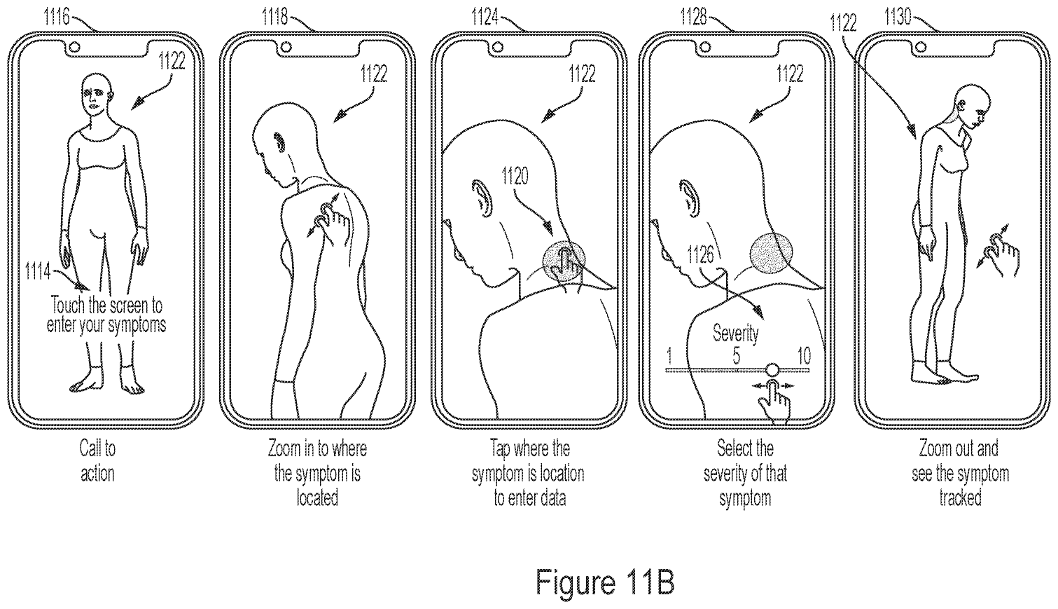

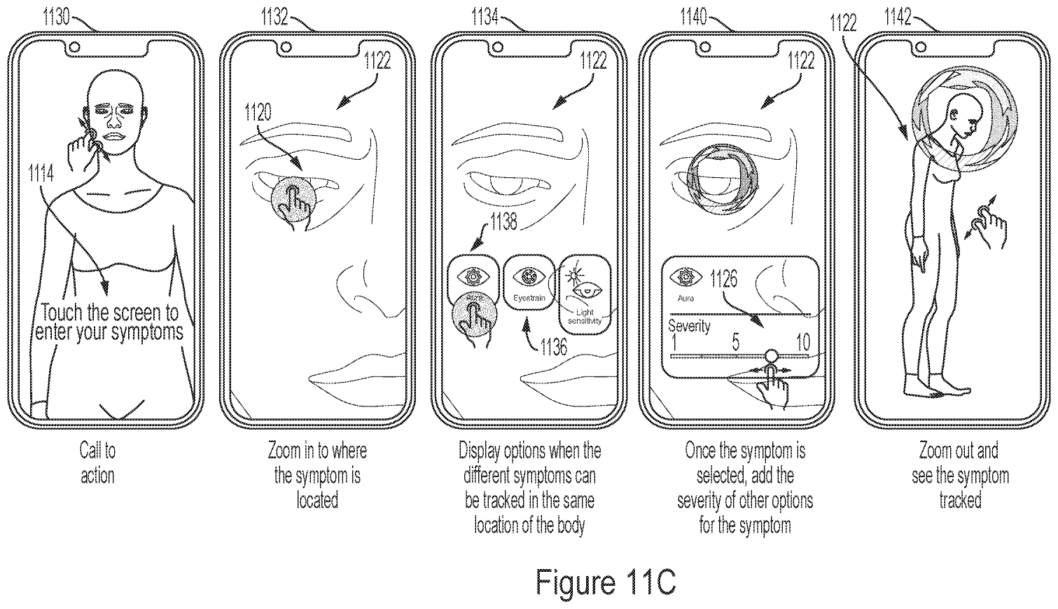

As illustrated by FIG. 11B, in an example embodiment, interacting with the patient avatar to enter patient data can include i) at 1116 a call to action 1114, such as waking up the patient avatar by touching a screen of the patient device on which the GUI is displayed, ii) at 1118 using touch to zoom in on a specific body area where a patient may be experiencing a disease symptom, iii) at 1124 selecting a symptomatic body part 1120 on the patient avatar 1122 via the GUI of the patient device, iv) at 1128 receiving a prompt to enter symptom data 1126 for the symptomatic body part, v) receiving the symptom data for the symptomatic body part at the server system associated with the patient device, and vi) at 1130 updating the patient avatar 1122 based on the symptom data. As illustrated in FIG. 11B at 1128, the prompt to enter the symptom data can be a sliding bar. By sliding the indicator on the bar, the color on the neck can change to indicate the severity of the symptom—neck pain. Alternatively, the texture, pattern, gradient, or shapes appearing on the neck could change. In an additional embodiment, the prompt to enter the symptom data can be a menu with different options to choose from. The options can include any of i) location of pain, ii) type of pain (e.g., throbbing vs. continuous), iii) severity, iv) frequency, v) start and end time of the disease symptom, or vi) specific notes on the disease symptom. Once a menu option is selected, the patient can use the sliding bar to indicate an answer, or the patient can type in a specific note.

As illustrated by FIG. 11C, in an example embodiment, interacting with the patient avatar to enter patient data can include i) at 1130 a call to action 1114, such as waking up the patient avatar by touching a screen of the patient device on which the GUI is displayed, ii) at 1132 using touch to zoom in on a specific body area where a patient may be experiencing a disease symptom and selecting a symptomatic body part 1120 on the patient avatar 1122 via the GUI of the patient device, iii) at 1134 when different disease symptoms can be related to one symptomatic body part, displaying a submenu 1136 and selecting at least one option 1138 from the submenu, iv) at 1140 entering the symptom data 1126 for the at least one option from the submenu, v) receiving the symptom data for the symptomatic body part at the server system associated with the patient device, and vi) at 1142 updating the patient avatar 1122 based on the symptom data. As illustrated in FIG. 11C at 1134, the submenu 1136 with at least one option can include different disease symptoms associated with the eye, such as aura, eyestrain, and light sensitivity. Once the symptom is selected, symptom data such as the quality or quantity of the symptom can be entered via a sliding bar in 1140. By sliding the indicator on the bar, the ex-avatar alert can appear on the symptomatic body part.

In an example embodiment, disease symptom data can also be entered vocally through voice recognition. Vocally entering the disease symptom data can be performed together or separately from entering data via texting and/or touch screen interactions with the GUI, as previously described. The patient device can be capable of recognizing vocal commands and can recognize at least one vocal activation word to begin the process of vocally entering disease symptoms. The method can also include receiving, via a microphone on the patient device, symptom data for at least one disease symptom. The patient can list symptomatic body parts and related disease symptoms that the patient is experiencing. The method can also include outputting, via a speaker on the patient device, an inquiry for additional symptom data to ensure that all of the disease symptom data has been reported. To indicate that all of the disease symptom data has been reported by the patient, the patient device can receive at least one vocal termination word from the patient. The patient avatar can then be updated based on the symptom data. In an example embodiment, the clinician may be able to enter symptom data on the patient avatar in a similar way.

In an example embodiment, once the disease symptom data is entered for a symptomatic body part, the GUI on the patient and/or clinician device can display at least one of i) a zoom in of the symptomatic body part, ii) a move to the symptomatic body party, iii) a tilt to show a different perspective of the symptomatic body part, or iv) a pan to the symptomatic body part. This can be done in an attempt to display the symptomatic body part and/or any associated texts or icons with the symptomatic body part.

At block 908, method 900 involves in response to receiving a playback command from the patient device, playing back a graphical representation of a temporal evolution of the patient avatar. The temporal evolution of the patient avatar can include a change over time in historical health information of the at least one patient, as previously discussed.

The systems and methods described herein generally relate to software application technology, and more particularly to healthcare management software. Software applications are typically most effective where users remain engaged and when chum rates are low. Similarly, healthcare management applications rely on consistent data from individuals with a patient population to arrive at meaningful conclusions. Accordingly, it is desirable within the software application and healthcare industry to promote user/patient engagement. One way of promoting user engagement is by providing beneficial recommendations based on data provided by the user in a visually appealing way. The above-described examples achieve this by receiving patient data, then generating and displaying a patient avatar based on the patient data, and by displaying the evolution of the patient data on the patient avatar. This manner of providing a visual aid that evolves similar to the patient establishes a direct relationship between user engagement and the patient avatar. In this way, a healthcare management application “rewards” users for engagement, and thus encourages continued use of the software. Accordingly, the described embodiments further drive user engagement by providing visualizations that are personalized for each patient. Thus, the systems and methods described herein provide specific implementations directed towards improving the technical fields of (i) software applications and (ii) healthcare management platforms.

For similar reasons, the systems and methods described herein effectuate improvements in computing systems associated with healthcare management. For example, the determined method may be used as an archive of massive amounts of patient data that can conveniently be updated and safely transferred between patients, clinicians, and other users. In this manner, computing requirements of the system can be improved by solving the problem of transferring large amounts of medical data safely and accurately. Still further, the visualization of the patient avatar and patient data can assist a clinician in quickly and accurately diagnosing patients in a virtual manner. These and other improvements to relevant technical fields will be understood to those having skill in the art.

While particular aspects and embodiments are disclosed herein, other aspects and embodiments will be apparent to those skilled in the art in view of the foregoing teaching. For example, while the embodiments and examples are described with respect to migraine headaches, the disclosed systems and methods are not so limited and can be applicable to a broad range of disease symptoms and related disease factors and disease triggers. The various aspects and embodiments disclosed herein are for illustration purposes only and are not intended to be limiting, with the true scope being indicated by the following claims.

1. A method of interacting with a patient avatar comprising:
receiving, at a centralized server system associated with at least one of a clinician device or a patient device, patient data for at least one patient, wherein the patient data comprises historical health information;
generating, based on the patient data for the at least one patient, the patient avatar, wherein the patient avatar comprises a two-dimensional or three-dimensional graphical representation of the at least one patient, and wherein the historical health information is visually displayed as physical characteristics of the patient avatar;
displaying, on a graphical user interface associated with at least one of the clinician device or the patient device, the patient avatar, wherein displaying the patient avatar comprises displaying, based on the historical health information, most recent patient data; and
in response to receiving a playback command from at least one of the clinician device or the patient device, playing back a graphical representation of a temporal evolution of the patient avatar, wherein the temporal evolution of the patient avatar comprises a change over time in historical health information of the at least one patient.

2. The method of claim 1, wherein the historical health information comprises at least one disease symptom and wherein the physical characteristics of the patient avatar are based on the at least one disease symptom of the at least one patient, wherein the physical characteristics of the patient avatar comprise an appearance of a symptomatic patient body part, wherein the appearance comprises at least one of: a size of the symptomatic patient body part, a color of the symptomatic patient body part, a shade of the symptomatic patient body part, a texture of the symptomatic patient body part, and an icon displayed on the symptomatic patient body part.

3. The method of claim 2, wherein playing back the graphical representation of the temporal evolution of the patient avatar comprises displaying changing disease symptoms of the patient.

4. The method of claim 1, wherein the historical health information is compiled over a temporal period.

5. The method of claim 1, further comprising:
prompting the patient avatar with a historical health information question; and
receiving an answer to the historical health information question, based on the historical health information.

6. The method of claim 1, wherein the historic health information comprises therapeutic intervention data.

7. The method of claim 1, wherein playing back the graphical representation of the temporal evolution of the patient avatar comprises displaying changes in historical health data from a previous clinician appointment to a current clinician appointment.

8. The method of claim 1, further comprising:
inputting a question command via the graphical user interface associated with the clinician device; and
in response to receiving the question command, displaying the question command on the graphical user interface associated with the patient device.

9. The method of claim 1, further comprising:
inputting a request for a real-time conversation via the graphical user interface associated with the clinician device; and
in response to receiving the request, displaying the request for a real-time conversation on the graphical user interface associated with the patient device, wherein the request includes at least an option to accept, deny, or schedule.

10. The method of claim 1, wherein the patient data for the at least one patient is gathered by a plurality of wearable devices worn by the patient.

11. The method of claim 1, wherein the patient avatar comprises a non-fungible token.

12. The method of claim 1, wherein the clinician device further comprises a clinician identity, and wherein the clinician identity comprises a clinician private key and a clinician public key.

13. The method of claim 12, wherein receiving patient data comprises receiving encrypted patient data by the clinician public key and decrypting the patient data by the clinician private key.

14. The method of claim 1, wherein the historical health information is transmitted from a patient identity and wherein the patient identity includes a patient private key and a patient public key.

15. The method of claim 14, further comprising:
sending the patient avatar from a patient public key to a recipient public key, wherein the recipient public key is associated with a recipient private key;
comparing the recipient private key to a database of private keys, wherein the database of private keys comprises a plurality of clinician private keys and a plurality of client private keys; and
based on a determination that the recipient private key is a clinician private key, sharing the patient avatar with the recipient private key.

16. The method of claim 15, further comprising determining that the recipient private key is not the clinician private key and based on the determination sharing a predetermined portion of the patient avatar with the recipient private key.

17. The method of claim 1, further comprising:
determining a trend in historical health information, wherein the trend is defined by health guidelines to be clinically significant; and
displaying, on the graphical user interface associated with at least one of the clinician device or the patient device, an alert of the trend, wherein displaying the alert comprises at least one of an in-avatar alert and an ex-avatar alert, wherein the in-avatar alert comprises changing avatar appearance, and wherein the ex-avatar alert comprises at least one of a symbol and a text box.

18. The method of claim 1, further comprising displaying, on the graphical user interface associated with at least one of the clinician device or the patient device, the patient avatar and at least one additional avatar, wherein the patient avatar and the additional avatar are displayed side-by-side on the graphical user interface.

19. The method of claim 18, wherein the patient avatar is generated based on the patient data from a first time period in the historical health information and wherein the additional avatar is generated based on the patient data for the at least one patient from a second time period in the historical health information.

20. The method of claim 18, wherein the additional avatar is generated based on additional patient data for at least one additional patient, wherein the additional avatar comprises a two-dimensional or three-dimensional graphical representation of the at least one additional patient, and wherein additional historical health information is visually displayed as physical characteristics of the additional avatar.

21. (canceled)

22. (canceled)

23. (canceled)

24. (canceled)

25. (canceled)

26. (canceled)

27. (canceled)
